(19) United States
(12) Patent Application Publication
(10) Pub. No.: US 2009/0155218 A1
(43) Pub. Date: Jun. 18, 2009

(54) COMPOSITIONS AND METHODS FOR GROWTH OF EMBRYONIC STEM CELLS

(76) Inventors: Alberto Hayek, La Jolla, CA (US); Gillian Beattie, San Diego, CA (US); Ana Lopez, San Diego, CA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 10/598,682

(22) PCT Filed: Mar. 9, 2005
(86) PCT No.: PCT/US05/07704

§ 371 Date: Jul. 30, 2007

Related U.S. Application Data
(60) Provisional application No. 60/552,318, filed on Mar. 10, 2004.

(51) Int. Cl. A61K 45/00; C12N 5/08; A61P 35/00

(52) U.S. Cl. 424/93.7; 435/374; 435/366

(57) ABSTRACT

The present invention relates to methods, compositions, and kits for maintenance of the undifferentiated state and/or pluripotency in stem cells, including embryonic stem (ES) cells. It also relates to stem cells that have been maintained in an undifferentiated state. The present invention provides these aspects using culture medium enriched with a TGFβ family member protein, an FGF family member protein, and/or nicotinamide, without using fibroblast feeder layers, conditioned media, or leukemia inhibitory factor.

### CROSS-REFERENCE TO RELATED APPLICATIONS

The present application claims the benefit of the filing date of U.S. provisional patent application No. 60/552,318, filed 10 Mar. 2004, the entire disclosure of which is hereby incorporated herein in its entirety by reference.

### BACKGROUND OF THE INVENTION

1. Field of the Invention

The present invention relates to the field of molecular and cellular biology. More particularly, the present invention relates to growth of embryonic stem cells in culture in an undifferentiated state, and use of such cells for the study of cellular processes and development of medically useful products.

2. Description of Related Art

Due to their pluripotency, stem cells show great promise for treatments of many human diseases. Their ability to differentiate into any cell type makes them a valuable resource for research and development of treatments for diseases and disorders affecting numerous different cell types, tissues, and organs. Thus, there is currently intense interest in studying them, developing methods for growing and maintaining them in an undifferentiated state, and controlling differentiation of them to produce cell types of interest for treatments.

It has been proposed that stem cells, including adult stem cells and embryonic stem cells, either as isolated or modified by introduction of new genes or deletion or replacement of defective genes, can be used to replace or supplement defective or lost body tissues and organs, including those that produce substances that are critical for health and life. However, maintaining undifferentiated pluripotent stem cells in culture, a necessity for this type of research and subsequently for treatment, is notoriously difficult. When placed in culture containing no supplemental additives, undifferentiated stem cells spontaneously begin rapid differentiation and then typically slow or stop dividing. Thus, to date, few stem cells are actually available for research and development of treatments. To overcome this problem, researcher are attempting to devise ways to culture stem cells in an undifferentiated state through addition of exogenous substances that inhibit differentiation while permitting the cells to continue to divide.

The two most widely studied embryonic stem cell types are mouse embryonic stem cells (mESC) and human embryonic stem cells (hESC). Maintenance of the undifferentiated state and pluripotency in mouse embryonic stem cells requires the presence of mouse fibroblast feeder layers (mEFs) or activation of STAT3 with leukemia inhibitory factor (LIF). Likewise, hESC are typically cultured on mEFs or in media obtained from growth of fibroblasts. Because human embryonic stem cell lines have only recently become available for research, the intracellular pathways for self renewal and differentiation are, at this time, largely unknown. However, it is becoming apparent that the requirements for growth of hESC will be significantly different from those required for growth of MESC. For example, recently it was discovered that, unlike the situation with mESC, activation of STAT3 is not sufficient to block differentiation of hESC when grown on mEFs or when treated with conditioned media from mEFs.⁴ In addition, in a report published after the filing date of the priority document for this application, it was disclose that basic fibroblast factor (bFGF), a type of keratinocyte growth factor (KGF), together with noggin, inhibits differentiation of hESC. (Xu, R.-H. et al., 2005). Thus, according to the authors, addition of bFGF to culture media enables the continued growth and maintenance of hESC without the need for feeder cells.

Although advances are being made in the culturing of stem cells, and specifically embryonic stem cells, there is still a need in the art for practical, reliable methods and materials for maintaining and growing undifferentiated stem cells in culture. In particular, there exists a need in the art for methods and materials for growth and maintenance of undifferentiated hESC.

### SUMMARY OF THE INVENTION

The present invention provides methods, compositions, and kits for growth and maintenance of undifferentiated stem cells, including embryonic stem cells. Thus, the present invention relates to maintenance of the undifferentiated state and/or pluripotency in embryonic stem cells. The methods, compositions, and kits are suitable for use in culturing stem cells for use in research, production of research and medical substances, and treatment of diseases and disorders, particularly those affecting humans.

In a first aspect, the present invention provides methods for growth and maintenance of undifferentiated stem cells. Generally, the methods comprise exposing stem cells to a member of the transforming growth factor β (TGFβ) family of proteins, such as Activin A, in an amount sufficient to maintain the cells in an undifferentiated state for a sufficient amount of time to achieve a desired result. The desired result can be any medically or scientifically relevant result, such as, but not limited to, production of a particular substance, confluency of the culture on a culture plate, production of a sufficient number of cells for transfer to new culture media (i.e., for passage), or production of a sufficient number of cells for implantation into a subject. The methods can comprise exposing the stem cells to a member of the fibroblast growth factor family (FGF) of proteins, such as KGF (also known as FGF7), in an amount sufficient to permit growth and maintenance of the cells in culture, including, but not limited to growth through multiple passages, such as about ten passages or more. Thus, the various forms of the method of the invention can result in growth and maintenance of stem cells in an undifferentiated state for at least ten passages in culture. The methods can likewise comprise exposing stem cells to nicotinamide (NIC) in an amount sufficient to permit growth and maintenance of the cells in culture through multiple passages, such as about ten passages, twenty passages, thirty passages, or more. The methods can permit growth and maintenance of stem cells in culture in the absence of any feeder cells, conditioned media, and/or leukemia inhibitory factor (LIF). In embodiments, the methods maintain the cells in a pluripotent state.

In a second aspect, the invention provides compositions that can be used to grow and/or maintain stem cells in an undifferentiated or pluripotent state. Generally, the compositions comprise a member of the TGFβ family, such as Activin A, a member of the FGF family, such as KGF, or NIC, or a combination of two or all three of these, in an amount and form that is sufficient to permit growth and/or maintenance of at least one culture of stem cells in an undifferentiated state for a sufficient amount of time to achieve a desired result. In accordance with the discussion of the methods, above, the desired result can be any medically or scientifically relevant result. Thus, in embodiments, the compositions can comprise KGF in amount sufficient to grow and/or maintain at least one culture of stem cells in an undifferentiated state for a sufficient amount of time to achieve a desired result. In embodiments, the compositions can comprise NIC in amount sufficient to grow and/or maintain at least one culture of stem cells in an undifferentiated state for a sufficient amount of time to achieve a desired result. In various embodiments, the compositions comprise combinations of two or more of Activin A, KGF, and NIC. The compositions of the invention generally comprise a component in addition to the TGFβ family member, the FGF family member, and/or NIC. The additional component can be any substance that is known to be suitable for, or compatible with, growth of stem cells or for introduction of stem cells or substances produced by stem cells into an animal or human subject. In embodiments, it is culture media. In view of the usefulness of the compositions of the invention in growing and maintaining stem cells, it is evident that the compositions of the invention can themselves comprise stem cells, including embryonic stem cells, such as hESC.

In a third aspect, the invention provides kits that contain some or all of the materials that are needed to grow and/or maintain embryonic stem cells in an undifferentiated or pluripotent state. In its most basic form, the kit comprises at least one container containing a member of the TGFβ family of proteins, a member of the FGF family of proteins, and/or NIC in an amount sufficient to grow and maintain stem cells in culture for a sufficient amount of time to achieve a desired result. The desired result can be any medically or scientifically relevant result, such as production of a detectable amount of a particular substance, confluency of the culture on a culture plate, production of a sufficient number of cells for implantation into a subject, and the like.

In a fourth aspect, the present invention provides stem cells that are undifferentiated and pluripotent. The stem cells are grown and maintained in the compositions of the invention. In embodiments, the stem cells are provided in compositions that do not comprise feeder cells, conditioned media from mouse embryonic feeder layers, and/or STAT3 activation.

### BRIEF DESCRIPTION OF THE DRAWINGS

The accompanying drawings, which are incorporated in and constitute a part of this specification, illustrate features of several embodiments of the invention, and together with the written description, serve to explain certain facets of the invention.

FIG. 1 shows differentiation of hES cells in the absence of activin A. FIG. 1a shows the morphology and differentiation state of HSF6 cells observed by phase contrast microscopy (upper layer), and immunohistochemistry (lower layer). FIG. 1b shows semi-quantitative RT-PCR (26 cycles) of hES cells for oct-4 and nanog under a variety of culture conditions on mEFs (lane 1) or on laminin (lane 2-5). FIG. 1c shows a representative experiment of comparison of cell surface-antigen expression using FACS. FIG. 1d shows a representative experiment of comparison of proliferation of hES cells in the presence of activin A (A), NIC(N), KGF (K) or a combination of all 3 (ANK), and in FGF2 supplemented CM.

FIG. 2 shows the effect of activin/follistatin on mEF maintenance of pluripotency in HSF6 cells. FIG. 2a shows HSF6 cells from FIG. 1 Panel I, cultured on mEFs in the presence of follistatin for 1 week (left panels) and 2 weeks (right panels). FIG. 2b shows semi-quantitative RT-PCR (26 cycles) of HSF6 cells on mEFs for oct-4 and nanog in the presence and absence of follistatin. FIG. 2c shows identification of activin A transcripts in mEFs derived from CF-1 mice and precursor protein in mEF conditioned media using RT-PCR and Western blots.

FIG. 2d shows identification of activin pathway signaling components in HSF6 cells.

FIG. 3 shows long-term maintenance of pluripotency in hES cells cultured with activin A NIC and KGF. FIG. 3a shows analysis of stem cell markers in HSF6 cells cultured in the presence of activin A, KGF and NIC for 20 passages.

FIG. 3b shows teratoma formation in nude mice. FIG. 3c shows RT-PCR analysis of lineage specific markers in embryoid bodies derived from hES cells cultured in the presence of activin A, KGF and NIC.

### DETAILED DESCRIPTION OF VARIOUS EMBODIMENTS OF THE INVENTION

Reference will now be made in detail to various exemplary embodiments of the invention. The following detailed description of certain embodiments of the invention should not be construed as limiting the invention in any way, but rather should be understood as an explanation of various embodiments to help describe the invention to those of skill in the art.

### DEFINITIONS

Unless otherwise specifically defined herein, all terms used in this document are used in accordance with their general and customary usage in the arts of molecular biology and cellular biology. The following terms are defined as follows:

As used herein, the words “a”, “an”, and “the” include both singular and plural references unless the context of the sentence clearly dictates otherwise.

As used herein, the term “or” means one or a combination of two or more of the listed choices.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not necessarily require sequential performance of the listed steps, unless the context of the sentence clearly dictates otherwise. For example, a method comprising steps a, b, and c encompasses a method of performing steps in the order of steps a, c, and b, the order of steps c, b, and a, and the order of steps c, a, and b, etc.

As used herein, the terms “contacting” and “exposing” are used interchangeably, and, when used in connection with cells and a substance, mean placing the substance in a location and under conditions that will allow it to touch the cell in order to produce “contacted” or “exposed” cells.

As used herein, the term “cell” refers to a single cell as well as to a population of (i.e., more than one) cells. The population may be a pure population comprising one cell type. alternatively, the population may comprise more than one cell type. In the present invention, there is no limit on the number of cell types that a cell population may comprise. Furthermore, as used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “mixed cell culture” refers to a mixture of two or more types of cells. In some embodiments, the cells are cell lines that are not genetically engineered, while in other embodiments the cells are genetically engineered cell lines. In some embodiments the cells contain genetically engineered molecules. The present invention, while not so limited, encompasses any combination of cell types suitable for production of teratomas, or for the detection, identification, and/or quantitation of apoptosis in samples, including mixed cell cultures in which all of the cell types used are not genetically engineered, mixtures in which one or more of the cell types are genetically engineered and the remaining cell types are not genetically engineered, and mixtures in which all of the cell types are genetically engineered.

As used herein, the term “primary cell” is a cell which is directly obtained from a tissue (e.g. blood) or organ of an animal, including a human, in the absence of culture. Typically, though not necessarily, a primary cell is capable of undergoing ten or fewer passages in vitro before senescence and/or cessation of proliferation. In contrast, a “cultured cell” is a cell which has been maintained and/or propagated in vitro for ten or more passages.

As used herein, the term “cultured cells” refer to cells which are capable of a greater number of passages in vitro before cessation of proliferation and/or senescence when compared to primary cells from the same source. Cultured cells include “cell lines” and “primary cultured cells.”

As used herein, the term “cell line” refers to cells that are cultured in vitro, including primary cell lines, finite cell lines, continuous cell lines, and transformed cell lines. The term does not require that the cells be capable of an infinite number of passages in culture. Cell lines may be generated spontaneously or by transformation.

As used herein, the terms “primary cell culture” and “primary culture” refer to cell cultures that have been directly obtained from cells in vivo, such as from animal or insect tissue. These cultures may be derived from adult as well as fetal tissue.

As used herein, the terms “monolayer”, “monolayer culture”, and “monolayer cell culture” refer to cells that have adhered to a substrate and grow as a layer that is one cell in thickness. Monolayer cells may be grown in any format, including but not limited to flasks, tubes, coverslips (e.g., shell vials), roller bottles, etc. Monolayer cells may also be grown attached to microcarriers, including but not limited to beads.

As used herein, the terms “suspension” and “suspension culture” refer to cells that survive and proliferate without being attached to a substrate. Suspension cultures can be produced using hematopoietic cells, transformed cell lines, and cells from malignant tumors.

As used herein, the terms “culture media” and “cell culture media” refer to media that are suitable to support the growth of cells in vitro (i.e., cell cultures). It is not intended that the term be limited to any particular culture medium. For example, it is intended that the definition encompass outgrowth as well as maintenance media. Indeed, it is intended that the term encompass any culture medium suitable for the growth of the cell cultures of interest.

As used herein the term, the term “in vitro” refers to an artificial environment and to processes or reactions that occur within an artificial environment. In vitro environments are exemplified by, but are not limited to, test tubes and cell cultures.

As used herein, the term “in vivo” refers to the natural environment (e.g., an animal or a cell) and to processes or reactions that occur within a natural environment.

As used herein, the terms “proliferation” and “growth”, which are used interchangeably, refer to an increase in cell number. In contrast, “maintenance” means continued survival of a cell or population of cells, but not necessarily survival with an increase in numbers of cells.

As used herein, the term “differentiate” and all of its forms refers to the maturation process cells undergo whereby they develop distinctive characteristics, and/or preform specific functions, and/or are less likely to divide.

As used herein, the terms “isolate” and “purify”, and all of their forms refer to the reduction in the amount of at least one contaminant (such as protein and/or nucleic acid sequence) from a sample. Thus purification results in an “enrichment” (i.e., an increase) in the amount of a desirable protein and/or nucleic acid sequence in the sample.

As used herein, the term “amino acid sequence” refers to an amino acid sequence of a naturally occurring or engineered protein molecule. “Amino acid sequence” and like terms, such as “polypeptide”, “peptide”, or “protein” are not meant to limit the amino acid sequence to the complete, native amino acid sequence associated with the recited protein molecule.

As used herein, the terms “receptor proteins” and “membrane receptor proteins” refer to membrane spanning proteins, or portions thereof, that bind a ligand (e.g., gp 130, a microbial molecule; endotoxin, such as LPS, LTA; dsRNA, and the like).

As used herein, the term “ligand” refers to a molecule that binds to a second molecule. A particular molecule may be referred to as either, or both, a ligand and second molecule. Examples of second molecules include a receptor of the ligand, and an antibody that binds to the ligand.

As used herein, the term “activating” when in reference to a biochemical response (such as kinase activity) and/or cellular response (such as cell proliferation) refers to increasing the biochemical and/or cellular response. As used herein, the term “activated” when in reference to a cell, refers to a cell that has undergone a response that alters its physiology and shifts it towards making a biologically response and becoming biologically “active” hence “activated”. For example, a monocyte becomes activated to mature into a macrophage. For another example, a macrophage becomes activated upon contact with endotoxin (such as LPS) wherein the activated macrophage can produce an increased level and/or type of molecule associated with activation (e.g. iNOS, MMP-12 Metalloelastase and the like). In another example, an immature dendritic cell becomes activated to mature into a functional dendritic cell. An “activated” cell does not necessarily, although it may, undergo growth or proliferation.

As used herein, the terms “naturally occurring”, “wild-type”, and “wt” when applied to a molecule or composition (such as nucleotide sequence, amino acid sequence, cell, apoptotic blebs, external phosphatidylserine, etc.), mean that the molecule or composition can be found in nature and has not been intentionally modified by man. For example, a naturally occurring polypeptide sequence refers to a polypeptide sequence that is present in an organism that can be isolated from a source in nature, wherein the polypeptide sequence has not been intentionally modified by man.

The terms “derived from” and “established from” when made in reference to any cell disclosed herein refer to a cell which has been obtained (e.g., isolated, purified, etc.) from the parent cell in issue using any manipulation, such as, without limitation, infection with virus, transfection with DNA sequences, treatment and/or mutagenesis using for example chemicals, radiation, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “biologically active,” refers to a molecule (e.g. peptide, nucleic acid sequence, carbohydrate molecule, organic or inorganic molecule, and the like) having structured, regulatory, and/or biochemical functions in vivo.

Unless defined otherwise in reference to the level of molecules and/or phenomena, the terms “reduce”, “inhibit”, “diminish”, “suppress”, “decrease”, and all of their forms, when in reference to the level of any molecule (e.g., protein, nucleic acid sequence, protein sequence, proliferation, rate of differentiation, etc.), phenomenon (e.g., protein-protein interactions, catalytic activity, apoptosis, cell death, cell survival, cell proliferation, cell differentiation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) in a first sample relative to a second sample, mean that the quantity of molecule and/or phenomenon in the first sample is lower than in the second sample by any amount that is detectable.

Unless defined otherwise in reference to the level of molecules and/or phenomena, the terms “increase”, “elevate”, “raise”, and all of their forms when in reference to the level of any molecule (e.g., protein, nucleic acid sequence, protein sequence, proliferation, rate of differentiation, etc.), phenomenon (e.g., protein-protein interactions, catalytic activity, apoptosis, cell death, cell survival, cell proliferation, cell differentiation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) in a first sample relative to a second sample, mean that the quantity of the molecule and/or phenomenon in the first sample is higher than in the second sample by any amount that is statistically significant using any art-accepted statistical method of analysis.

As used herein, the term “apoptosis” refers to the process of non-necrotic death that takes place in metazoan animal cells following activation of an intrinsic cell suicide program. Apoptosis is a normal process in the proper development and homeostasis of metazoan animals and usually leads to cell death. Apoptosis is also triggered pathologically by microbial infections resulting in increasing susceptibility to apoptosis and/or outright death. Apoptosis involves sequential characteristic morphological and biochemical changes. One early marker of apoptosis is the flipping of plasma membrane phosphatidylserine, inside to outside, with cellular blebbing called “zeiosis,” of plasma membrane releasing vesicles containing cellular material including RNA and DNA as apoptotic bodies. During apoptosis, there is cell expansion followed by shrinkage through release of apoptotic bodies and lysis of the cell, nuclear collapse and fragmentation of the nuclear chromatin, at certain intranucleosomal sites, due to activation of endogenous nucleases. Apoptotic bodies are typically phagocytized by other cells, in particular immunocytes such as monocytes, macrophages, immature dendritic cells and the like. One of skill in the art appreciates that reducing the ability to undergo apoptosis results in increased cell survival, without necessarily (although it may include) increasing cell proliferation. Accordingly, as used herein, the terms “reduce apoptosis” and “increase survival” are equivalent. Also, as used herein, the terms “increase apoptosis” and “reduced survival” are equivalent.

As used herein, the term “cellular response” refers to an increase or decrease of activity by a cell. For example, the “cellular response” may constitute but is not limited to apoptosis, death, DNA fragmentation, blebbing, proliferation, differentiation, adhesion, migration, DNA/RNA synthesis, gene transcription and translation, and/or cytokine secretion or cessation of such processes. A “cellular response” may comprise an increase or decrease of dephosphorylation, phosphorylation, calcium flux, target molecule cleavage, protein-protein interaction, nucleic acid-nucleic acid interaction, and/or protein/nucleic acid interaction and the like. As used herein, the term “target molecule cleavage” refers to the splitting of a molecule (for example in the process of apoptosis, cleavage of procaspases into fragments, cleavage of DNA into predicable sized fragments and the like). As used herein, the term “interaction” refers to the reciprocal action or influence of two or more molecules on each other.

As used herein, the term “transgenic” when used in reference to a cell refers to a cell that contains a transgene, or whose genome has been altered by the introduction of a transgene. The term “transgenic” when used in reference to a tissue refers to a tissue that comprises one or more cells that contain a transgene, or whose genome has been altered by the introduction of a transgene. Transgenic cells and tissues may be produced by several methods including the introduction of a “transgene” comprising nucleic acid (usually DNA) into a target cell or integration of the transgene into a chromosome of a target cell by way of human intervention, such as those known to those of skill in the art.

As used herein, the term “transgene” refers to any nucleic acid sequence which is introduced into the cell by experimental manipulations. A transgene may be an “endogenous DNA sequence” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

As used herein, the terms “agent”, “test agent”, “molecule”, “test molecule”, “compound”, and “test compound” are used interchangeably herein and refer to any type of molecule (for example, a peptide, nucleic acid, carbohydrate, lipid, organic molecule, and inorganic molecule, etc.) any combination molecule for example glycolipid, etc.) obtained from any source (for example, plant, animal, protist, and environmental source etc.), or prepared by any method (for example, purification of naturally occurring molecules, chemical synthesis, and genetic engineering methods, etc.). Thus, these terms are synonymous with the term “substance”. In one embodiment, the term “test agent” refers to any chemical entity, pharmaceutical, drug, and the like that can be used to treat or prevent a disease, illness, sickness, or disorder of bodily function. Test agents comprise both known and potential therapeutic agents. A test agent can be determined to be therapeutic by screening using the screening methods of the present invention. A “known therapeutic agent” refers to a therapeutic agent that has been shown (e.g., through animal trials or prior experience with administration to humans) to be effective in such treatment or prevention. In other words, a known therapeutic agent is not limited to an agent efficacious in the treatment of disease (e.g., cancer). Agents are exemplified by, but not limited to, antibodies, nucleic acid sequences such as ribozyme sequences, and other agents as further described herein. The test agents identified by and/or used in the invention's methods include any type of molecule (for example, a peptide, nucleic acid, carbohydrate, lipid, organic, and inorganic molecule, etc.) obtained from any source (for example, plant, animal, and environmental source, etc.), or prepared by any method (for example, purification of naturally occurring molecules, chemical synthesis, and genetic engineering methods, etc.).

Unless otherwise indicated by the terms “exactly”, “precisely”, or another equivalent term, all numbers expressing quantities of ingredients, properties such as molecular weight, reaction conditions, and so forth as used herein, are to be understood as being modified in all instances by the term “about”, and thus to inherently include variations of up to 10% greater or less than the actual number stated. Accordingly, unless indicated to the contrary, the numerical parameters herein are approximations that may vary depending upon the desired properties sought to be obtained by the present invention. At the very least, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “alter” and all of its forms as used herein in reference to the level of any molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) refers to an increase or decrease in the quantity of the molecule and/or phenomenon, regardless of whether the quantity is determined objectively, and/or subjectively.

Reference herein to any specifically named protein (such as activin A polypeptide, KGF, etc.) refers to any and all equivalent fragments, fusion proteins, and variants of the specifically named protein, having at least one of the biological activities (disclosed herein) of the specifically named protein, wherein the biological activity is detectable by any method. Naming of a protein thus includes all forms of that protein, including specific forms of a protein that is generically referred to herein.

The term “fragment” when in reference to a protein (such as activin A polypeptide, KGF, etc.) refers to a portion of that protein that may range in size from four (4) contiguous amino acid residues to the entire amino acid sequence minus one amino acid residue. Thus, a polypeptide sequence comprising “at least a portion of an amino acid sequence” is equivalent to a “fragment” and comprises from four (4) contiguous amino acid residues of the amino acid sequence to the entire amino acid sequence.

The terms “variant” and “homolog” (such as activin A polypeptide, KGF, etc.) as used herein are proteins that differ from a reference protein by insertion, deletion, and/or substitution of one or more amino acids, where the insertion, deletion, and/or substitution preferably does not alter the primary biological function of the reference protein. Variants and homologs include proteins having structural and functional characteristics in common with a reference protein, but from a different species. In embodiments, the substitutions are conserved changes to one or more amino acids. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine my be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. Thus, members of a family of proteins, such as members of the TGFβ family and the FGF family, contain variants and homologs of other members of the family.

The “TGFβ family” means proteins having structural and functional characteristics of known TGFβ family members. The TGFβ family of proteins is well characterized, both from structural and functional aspects. It includes the TGFβ series of proteins, the Inhibins (including Inhibin A and Inhibin B), the Activins (including Activin A, Activin B, and Activin AB), MIS (Müllerian inhibiting substance), BMP (bone morphogenetic proteins), dpp (decapentaplegic), Vg-1, MNSF (monoclonal nonspecific suppressor factor), and others. Of the various well-characterized activities of the TGFβ family members, TGFβ is considered to be the most potent growth inhibitor for normal and transformed epithelial cells, endothelial cells, fibroblasts, neuronal cells, lymphoid cells, and other hematopoietic cell types. Activity of this family of proteins is based on specific binding to certain receptors on various cell types. Members of this family share regions of sequence identity, particularly at the C-terminus, that correlate to their function. The TGFβ family includes more than one hundred distinct proteins, all sharing at least one region of amino acid sequence identity. Members of the family include, but are not limited to, the following proteins, as identified by their GenBank accession numbers: P07995, P18331, P08476, Q04998, P03970, P43032, P55102, P27092, P42917, P09529, P27093, P04088, Q04999, P17491, P55104, Q9WUK5, P55103, O88959, O08717, P58166, O61643, P35621, P09534, P48970, Q9NR23, P25703, P30884, P12643, P49001, P21274, O46564, O19006, P22004, P20722, Q04906, Q07104, P30886, P18075, P23359, P22003, P34821, P49003, Q90751, P21275, Q06826, P30885, P34820, Q29607, P12644, Q90752, O46576, P27539, P48969, Q26974, P07713, P91706, P91699, P27091, O42222, Q24735, P20863, O18828, P55106, Q9PTQ2, O14793, O08689, O42221, O18830, O18831, O18836, O35312, O42220, P43026, P43027, P43029, O95390, Q9R229, O93449, Q9Z1W4, Q9BDW8, P43028, Q7Z4P5, P50414, P17246, P54831, P04202, P01137, P09533, P18341, O19011, Q9Z1Y6, P07200, Q9Z217, O95393, P55105, P30371, Q9MZE2, Q07258, Q96S42, P97737, AAA97415.1, NP_{—}776788.1, NP_{—}058824.1, EAL24001.1, 1S4Y, NP_{—}001009856.1, NP_{—}032406.1, NP_{—}999193.1, XP_{—}519063.1, AAG17260.1, CAA40806.1, NP_{—}001009458.1, AAQ55808.1, AAK40341.1, AAP33019.1, AAK21265.1, AAC59738.1, CAI46003.1, B40905, AAQ55811.1, AAK40342.1, XP_{—}540364.1, P55102, AAQ55810.1, NP_{—}990727.1, CAA51163.1, AAD50448.1, JC4862, PN0504, BAB17600.1, AAH56742.1, BAB17596.1, CAG06183.1, CAG05339.1, BAB17601.1, CAB43091.1, A36192, AAA49162.1, AAT42200.1, NP_{—}789822.1, AAA59451.1, AAA59169.1, XP_{—}541000.1, NP_{—}990537.1, NP_{—}002184.1, AAC14187.1, AAP83319.1, AAA59170.1, BAB16973.1, AAM66766.1, WFPGBB, 1201278C, AAH30029.1, CAA49326.1, XP_{—}344131.1, AAH48845.1, XP_{—}148966.3, I48235, B41398, AAH77857.1, AAB26863.1, 1706327A, BAA83804.1, NP_{—}571143.1, CAG00858.1, BAB17599.1, BAB17602.1, AAB61468.1, PN0505, PN0506, CAB43092.1, BAB17598.1, BAA22570.1, BAB16972.1, BAC81672.1, BAA12694.1, BAA08494.1, B36192, C36192, BAB16971.1, NP_{—}034695.1, AAA49160.1, CAA62347.1, AAA49161.1, AAD30132.1, CAA58290.1, NP_{—}005529.1, XP_{—}522443.1, AAM27448.1, XP_{—}538247.1, AAD30133.1, AAC36741.1, AAH10404.1, NP_{—}032408.1, AAN03682.1, XP_{—}509161.1, AAC32311.1, NP_{—}651942.2, AAL51005.1, AAC39083.1, AAH85547.1, NP_{—}571023.1, CAF94113.1, EAL29247.1, AAW30007.1, AAH90232.1, A29619, NP_{—}001007905.1, AAH73508.1, AAD02201.1, NP_{—}999793.1, NP_{—}990542.1, AAF19841.1, AAC97488.1, AAC60038.1, NP 989197.1, NP_{—}571434.1, EAL41229.1, AAT07302.1, CAI19472.1, NP_{—}031582.1, AAA40548.1, XP_{—}535880.1, NP_{—}037239.1, AAT72007.1, XP_{—}418956.1, CAA41634.1, BAC30864.1, CAA38850.1, CAB81657.2, CAA45018.1, CAA45019.1, BAC28247.1, NP_{—}031581.1, NP_{—}990479.1, NP_{—}999820.1, AAB27335.1, S45355, CAB82007.1, XP_{—}534351.1, NP_{—}058874.1, NP_{—}031579.1, 1REW, AAB96785.1, AAB46367.1, CAA05033.1, BAA89012.1, 1ES7, AAP20870.1, BAC24087.1, AAG09784.1, BAC06352.1, AAQ89234.1, AAM27000.1, AAH30959.1, CAG01491.1, NP_{—}571435.1, 1REU, AAC60286.1, BAA24406.1, A36193, AAH55959.1, AAH54647.1, AAH90689.1, CAG09422.1, BAD16743.1, NP_{—}032134.1, XP_{—}532179.1, AAB24876.1, AAH57702.1, AAA82616.1, CAA40222.1, CAB90273.2, XP_{—}342592.1, XP_{—}534896.1, XP_{—}534462.1, 1LXI, XP_{—}417496.1, AAF34179.1, AAL73188.1, CAF96266.1, AAB34226.1, AAB33846.1, AAT12415.1, AAO33819.1, AAT72008.1, AAD38402.1, BAB68396.1, CAA45021.1, AAB27337.1, AAP69917.1, AAT12416.1, NP_{—}571396.1, CAA53513.1, AAO33820.1, AAA48568.1, BAC02605.1, BAC02604.1, BAC02603.1, BAC02602.1, BAC02601.1, BAC02599.1, BAC02598.1, BAC02597.1, BAC02595.1, BAC02593.1, BAC02592.1, BAC02590.1, AAD28039.1, AAP74560.1, AAB94786.1, NP_{—}001483.2, XP_{—}528195.1, NP_{—}571417.1, NP_{—}001001557.1, AAH43222.1, AAM33143.1, CAG10381.1, BAA31132.1, EAL39680.1, EAA12482.2, P34820, AAP88972.1, AAP74559.1, CAI16418.1, AAD30538.1, XP_{—}345502.1, NP_{—}038554.1, CAG04089.1, CAD60936.2, NP_{—}031584.1, B55452, AAC60285.1, BAA06410.1, AAH52846.1, NP_{—}031580.1, NP_{—}036959.1, CAA45836.1, CAA45020.1, Q29607, AAB27336.1, XP_{—}547817.1, AAT12414.1, AAM54049.1, AAH78901.1, AAO25745.1, NP_{—}570912.1, XP_{—}392194.1, AAD20829.1, AAC97113.1, AAC61694.1, AAH60340.1, AAR97906.1, BAA32227.1, BAB68395.1, BAC02895.1, AAW51451.1, AAF82188.1, XP_{—}544189.1, NP_{—}990568.1, BAC80211.1, AAW82620.1, AAF99597.1, NP_{—}571062.1, CAC44179.1, AAB97467.1, AAT99303.1, AAD28038.1, AAH52168.1, NP_{—}001004122.1, CAA72733.1, NP_{—}032133.2, XP_{—}394252.1, XP_{—}224733.2, JH0801, AAP97721.1, NP_{—}989669.1, S43296, P43029, A55452, AAH32495.1, XP_{—}542974.1, NP_{—}032135.1, AAK30842.1, AAK27794.1, BAC30847.1, EAA12064.2, AAP97720.1, XP_{—}525704.1, AAT07301.1, BAD07014.1, CAF94356.1, AAR27581.1, AAG13400.1, AAC60127.1, CAF92055.1, XP_{—}540103.1, AAO20895.1, CAF97447.1, AAS01764.1, BAD08319.1, CAA10268.1, NP_{—}998140.1, AAR03824.1, AAS48405.1, AAS48403.1, AAK53545.1, AAK84666.1, XP_{—}395420.1, AAK56941.1, AAC47555.1, AAR88255.1, EAL33036.1, AAW47740.1, AAW29442.1, NP_{—}722813.1, AAR08901.1, AAO15420.2, CAC59700.1, AAL26886.1, AAK71708.1, AAK71707.1, CAC51427.2, AAK67984.1, AAK67983.1, AAK28706.1, P07713, P91706, P91699, CAG02450.1, AAC47552.1, NP_{—}005802.1, XP_{—}343149.1, AW34055.1, XP_{—}538221.1, AAR27580.1, XP_{—}125935.3, AAF21633.1, AAF21630.1, AAD05267.1, Q9Z1W4, NP_{—}031585.2, NP_{—}571094.1, CAD43439.1, CAF99217.1, CAB63584.1, NP_{—}722840.1, CAE46407.1, XP_{—}417667.1, BAC53989.1, BAB19659.1, AAM46922.1, AAA81169.1, AAK28707.1, AAL05943.1, AAB17573.1, CAH25443.1, CAG10269.1, BAD16731.1, EAA00276.2, AAT07320.1, AAT07300.1, AAN15037.1, CAH25442.1, AAK08152.2, 2009388A, AAR12161.1, CAG01961.1, CAB63656.1, CAD67714.1, CAF94162.1, NP_{—}477340.1, EAL24792.1, NP_{—}001009428.1, AAB86686.1, AAT40572.1, AAT40571.1, AAT40569.1, NP_{—}033886.1, AAB49985.1, AAG39266.1, Q26974, AAC77461.1, AAC47262.1, BAC05509.1, NP_{—}055297.1, XP_{—}546146.1, XP_{—}525772.1, NP_{—}060525.2, AAH33585.1, AAH69080.1, CAG12751.1, AAH74757.2, NP_{—}034964.1, NP_{—}038639.1, O42221, AAF02773.1, NP_{—}062024.1, AAR18244.1, AAR14343.1, XP_{—}228285.2, AAT40573.1, AAT94456.1, AAL35278.1, AAL35277.1, AAL17640.1, AAC08035.1, AAB86692.1, CAB40844.1, BAC38637.1, BAB16046.1, AAN63522.1, NP_{—}571041.1, AAB04986.2, AAC26791.1, AAB95254.1, BAA11835.1, AAR18246.1, XP_{—}538528.1, BAA31853.1, AAK18000.1, XP_{—}420540.1, AAL35276.1, AAQ98602.1, CAE71944.1, AAW50585.1, AAV63982.1, AAW29941.1, AAN87890.1, AAT40568.1, CAD57730.1, AAB81508.1, AAS00534.1, AAC59736.1, BAB79498.1, AAA97392.1, AAP85526.1, NP_{—}999600.2, NP_{—}878293.1, BAC82629.1, CAC60268.1, CAG04919.1, AAN10123.1, CAA07707.1 AAK20912.1, AAR88254.1, CAC34629.1, AAL35275.1, AAD46997.1, AAN03842.1, NP_{—}571951.2, CAC50881.1, AAL99367.1, AAL49502.1, AAB71839.1, AAB65415.1, NP_{—}624359.1, NP_{—}990153.1, AAF78069.1, AAK49790.1, NP_{—}919367.2, NP_{—}001192.1, XP_{—}544948.1, AAQ18013.1, AAV38739.1, NP_{—}851298.1, CAA67685.1, AAT67171.1, AAT37502.1, AAD27804.1, AAN76665.1, BAC11909.1, XP_{—}421648.1, CAB63704.1, NP_{—}037306.1, A55706, AAF02780.1, CAG09623.1, NP_{—}067589.1, NP_{—}035707.1, AAV30547.1, AAP49817.1, BAC77407.1, AAL87199.1, CAG07172.1, B36193, CAA33024.1, NP_{—}001009400.1, AAP36538.1, XP_{—}512687.1, XP_{—}510080.1, AAH05513.1, 1KTZ, AAH14690.1, AAA31526.1.

The “FGF family” means proteins having structural and functional characteristics of known FGF family members. The FGF family of proteins is well characterized, both from structural and functional aspects. It includes at least 20 distinct members (not including variants and homologs), including the specific FGF series of proteins (FGF1 and FGF2), the K-FGF (found in Kaposi's sarcoma cells), and the KGF proteins. The members of this family that have been identified to date exhibit 30-70% amino acid sequence homology. Members of the family include, but are not limited to, the following proteins, as identified by their GenBank accession numbers: P21781, P79150, P48808, Q9N198, P36363, Q02195, P70492, O15520, O35565, Q9ESS2, Q9HCT0, P36364, P54130, P31371, O43320, Q91875, Q9NP95, O54769, P48801, P36386, P05524, P11487, P48802, Q92915, P12034, P48807, P15656, P70379, P48806, Q8R5L7, P48805, P70377, P48804, Q92913, P61329, Q11184, P10767, Q92914, P21658, P70378, P08620, P61148, Q616M7, P48800, P34004, P15655, P13109, P09038, P48803, Q6PBT8, P05230, P03969, Q7SIF8, P20003, P48798, P12226, Q6GLR6, P20002, P03968, Q7M303, P19596, Q60487_{—}3, Q6SJP8, P48799, O76093, O89101, O88182, P11403, O60258, P63075, P37237, Q9N1S8, P55075, Q90722, Q805B2, O35622, O95750, Q9JJN1, Q9NSA1, Q9GZV9, P41444, O10284, Q9EPC2, Q8VI82, O62682, Q9JYA0, Q9JT82, P01030_{—}3, NP_{—}002000.1, AAA67335.1, AAX19003.1, NP_{—}001003237.1, NP_{—}001009235.1, NP_{—}032034.1, S26049, AAF26734.1, BAC39707.1, NP_{—}071518.1, AAL16059.1, AAG31597.1, BAD84165.1, AAH88532.1, AAR87872.1, B46289, C46289, NP_{—}001007762.1, CAB90393.1, D46289, NP_{—}004456.1, XP_{—}526931.1, NP_{—}037083.1, BAB60779.1, AAM46926.1, CAG46489.1, NP_{—}032028.1, BAD74123.1, AAL05875.1, AAK59700.1, NP_{—}001009230.1, AAR37413.1, NP_{—}990027.1, CAB76368.1, CAD29182.1, AAC78789.1, CAG08586.1, NP_{—}878290.1, AAL16959.1, NP_{—}570107.1, NP_{—}075793.1, AAH10956.1, BAC57976.1, AAQ93357.1, AAC25096.1, AAL16963.1, AAO25617.1, AAG29501.1, NP_{—}989730.1, NP_{—}998966.1, CAC17692.1, NP_{—}038546.1, NP_{—}037084.1, NP_{—}003859.1, XP_{—}420304.1, NP_{—}068639.1, BAB71729.1, AAT85804.1, NP_{—}085117.1, CAF99081.1, NP_{—}076451.1, NP_{—}085113.1, BAC34892.1, CAF91044.1, XP_{—}426335.1, CAA87635.1, CAG13262.1, CAA80987.1, AAH81367.1, CAG01370.1, NP_{—}032033.1, NP_{—}570830.1, NP_{—}571366.1, NP_{—}005238.1, AAC06148.1, BAC22069.1, XP_{—}529049.1, AAH76721.1, NP_{—}001001743.1, AAF31398.1, AAO33291.1, AAL16957.1, CAA44480.1, NP_{—}991265.1, AAN16025.1, AAB18918.1, CAI15768.1, NP_{—}034330.2, NP_{—}990108.1, NP_{—}445880.1, AAF31392.1, AAB71606.1, P70379, XP_{—}542656.1, NP_{—}071559.2, NP_{—}997550.1, CAA44479.1, AAL83904:1, NP_{—}001012382.1, NP_{—}001009561.1, AAW69551.1, AAN04097.1, TVHUF5, NP_{—}004455.1, P12034, NP_{—}378668.1, NP_{—}004105.1, AAH85439.1, CAI43189.1, CAI42700.1, CAI42699.1, BAD72887.1, CAG09626.1, NP_{—}001009563.1, XP_{—}534534.1, XP_{—}524019.1, NP_{—}001012380.1, NP_{—}071547.1, NP_{—}034333.1, NP_{—}990219.1, AAF31390.1, XP_{—}549094.1, CAA76422.1, CAG07674.1, NP_{—}004104.3, XP_{—}526425.1, XP_{—}535845.1, XP 521287.1, NP_{—}570827.1, CAH91802.1, AAH22524.1, P48804, AAB71607.1, AAV90630.1, CAF98890.1, AAB53825.2, CAH93046.1, CAG01557.1, H88481, JG0184, AAB18786.3, CAA94240.1, NP_{—}004103.1, XP_{—}511297.1, CAD19830.1, AAC98812.1, BAB88673.1, NP_{—}066276.2, XP_{—}543862.1, CAA45054.1, CAI51960.1, XP_{—}546591.1, XP_{—}485825.1, NP_{—}570829.1, CAG07288.1, CAA35925.1, CAA94239.1, BAC39686.1, BAB63249.1, AAA62261.1, 2020426A, NP_{—}571983.1, NP_{—}571710.1, CAG04681.1, CAG09818.1, CAF99854.1, AAL16961.1, CAF99073.1, CAB37648.2, AAP32155.1, 1BFG, 1IIL, 1FQ9, 1BLD, 1BFC, 1BAS, AAH37601.1, BAD24666.1, NP_{—}957054.1, A60721, CAG11711.1, 1K5V, NP_{—}001998.1, 1JY0, BAD69615.1, CAG00679.1, AAH32697.1, CAI29610.1, BAC22072.1, AAB29057.2, 1JQZ, 1605206A, CAI43097.1, CAE72484.1, 11JT, A48834, NP_{—}001001398.1, NP_{—}990764.1, AAH74391.1, 1P63, CAF94666.1, 1JTC, AAA52534.1, A32398, P09038, AAQ73204.1, AAV74297.1, NP_{—}001997.4, AAV70487.1, AAA52532.1, 1JT4, 1JT3, 1BFF, 2BFH, NP_{—}062178.1, 1E0O, XP_{—}526572.1, NP_{—}032032.1, 1HKN, 1DZD, 1DZC, 1EVT, 2AXM, XP_{—}544946.1, AAL82819.1, Q7SIF8, 1JT5, P48803, CAA42869.1, 11PZZ, 1M16, S00185, P48798, NP_{—}776481.1, NP_{—}001009769.1, AAV67380.1, XP_{—}533298.1, A40117, 1Q03, GKBOB, AAC35912.1, CAA78854.1, JC4268, S31622, BAB40835.1, AAA72209.1, BAA89483.1, AAA57275.1, 1JT7, CAA46341.1, AAH74324.1, 1Q04, BAC38631.1, 1NZK, CAG02426.1, 1K5U, 1DJS, NP_{—}776480.1, NP_{—}034332.1, BAC22070.1, CAG06239.1, 1AFC, BAC22066.1, 1201195A, CAA43863.1, Q7M303, NP_{—}999973.1, XP_{—}522091.1, NP_{—}990511.1, CAG02165.1, AAK52308.1, BAC22067.1, Q60487_{—}3, XP 426414.1, XP_{—}540801.1, 1BAR, AAV38378.1, NP_{—}032031.1, NP_{—}062072.1, P48799, NP_{—}990045.1, AAK37962.1, CAG04671.1, BAA13958.1, XP_{—}544322.1, NP_{—}446261.1, AAQ89228.1, NP_{—}003858.1, XP_{—}522325.1, XP_{—}528080.1, NP_{—}062071.1, BAC38656.1, AAF75524.1, AAO38854.1, BAD74124.1, XP_{—}396434.1, BAD72875.1, AAP22372.1, CAF90784.1, AAC60303.1, AAF73225.1, CAF90913.1, AAP92385.1, BAB68397.1, NP_{—}034335.1, AAF73226.1, AAH86718.1, CAA71365.1, AAO15593.1, AAG45674.1, AAB82614.1, AAA93298.1, AAM22684.1, Q90722, CAF91385.1, NP_{—}149354.1, NP_{—}579820.1, AAB34255.1, AAM55238.1, AAH48734.1, AAH69106.1, AAN73036.1, BAC02894.1, AAH82344.1, CAB64349.1, AAK52310.1, AAK52309.1, AAA48616.1, AAL16958.1, XP_{—}543982.1, AAO25618.1, CAE11791.1, CAF95430.1, AAD13853.1, EAL29203.1, NP_{—}990005.1, XP 543258.1, XP_{—}547883.1, XP_{—}527117.1, NP_{—}878276.1, BAC03477.1, CAC86028.1, NP_{—}001012379.1, NP_{—}732452.1, NP_{—}732453.1, AAO41473.1, AAC47427.1, NP_{—}001012246.1, XP_{—}522624.1, AAQ89954.1, AAH66859.1, NP_{—}032029.1, YP_{—}164239.1, XP_{—}549294.1, 1PWA, BAD06584.1, AAF70374.1, NP_{—}570109.1, AAQ88669.1, AAP36636.1, YP_{—}195014.1, CAG06656.1, NP_{—}570108.1, BAA85130.1, AAQ89444.1, XP_{—}524333.1, NP_{—}064397.1, AAH18404.1, NP_{—}061986.1, BAA85129.1, NP_{—}001009564.2, BAD69717.1, BAC22071.1, AAN28104.1, AAQ88689.1, AAF65566.1, AAB71608.1, XP_{—}540802.1, CAI25614.1, AAC59026.1, AAD15898.1, AAA85394.1, CAG06655.1, CAA41788.1, AAA66662.1, XP_{—}541510.1, CAG01371.1, BAD02829.1, BAC22068.1, XP_{—}425663.1, BAC55965.1, NP_{—}073148.1, XP_{—}421720.1, BAD60785.1, AAC63708.1, O62682, XP_{—}522092.1, NP_{—}570110.1, XP_{—}392106.1, CAG02425.1, AAV97593.1, BAB68346.1, AAL16962.1, YP_{—}164240.1, YP_{—}195015.1, NP_{—}848339.1, CAF99321.1, CAA71967.1, A32484, CAA28029.1, AAL01804.1, AAK85589.1, AAM93422.1, CAA05888.1, AAO27576.1, AAO27575.1, CAH93705.1, AAU00991.1, AAL37368.1, EAL46196.1, NP_{—}701691.1.

The term “teratoma” refers to a tumor arising from cells of the three embryologic germ cell layers: ectoderm, mesoderm, and endoderm. The term “embryologic germ cell layers” as used herein refers to layers of cells in the embryo that become specialized and express certain characteristic features in their final developed form (e.g. ectoderm, mesoderm, and endoderm). The term “endoderm” refers to cells of the endodermal germ layers that develop into intestines. The term “mesoderm” refers to cells of the mesodermal germ layers that develop into blood vessels. The term “ectoderm” refers to cells of the ectodermal germ layers that develop into central and peripheral nerves, epidermis of skin.

The terms “stem cell”, “unspecialized cell”, “uncommitted cell”, and “undifferentiated cell” refer to a cell that has a unique capacity to renew itself and to give rise to specialized cell types that make up the tissues and organs of the body. A stem cell is without tissue-specific structures and tissue-specific functions (e.g. heart muscle cell, nerve cell, etc.). Stem cells can be derived from embryonic (e.g. embryonic stem cell), fetal, and adult tissues. The terms “specialized”, “committed”, and “differentiated” refer to cells with tissue-specific structures and/or tissue-specific functions (e.g. heart muscle cell, nerve cell, etc.). The term “differentiation” when it refers a cell refers to the process whereby an unspecialized acquires the features of a specialized cell (e.g. a heart, liver, or muscle cell).

The term “progenitor cell” refers to a cell in fetal and/or adult tissues and is partially specialized, can divide, and gives rise to differentiated cells.

The terms “embryonic stem cell”, “ES cell”, and “pluripotent cell” refer to undifferentiated cells derived from the inner cell mass of embryos that have the potential to become any specialized cell type. The term “embryonic germ cell” refers to a cell derived of fetal tissue including, for example, from the primordial germ cells of the gonadal ridge of the 5 to 10 week fetus.

The term “mammalian embryonic stem cell” refers to ES cells derived from a mammal. It is not meant to limit the mammals that can contribute stem cells, and thus can include humans (hES), monkeys, great apes, pigs, horses, cows, sheep, dogs, cats, mice, rats, etc.

The terms “adult stem cell”, “multipotent stem cell”, and “somatic stem cell” refer to an undifferentiated cell found in a differentiated tissue that can proliferate and differentiate to yield the specialized cell types of the tissue from which it originated.

The term “clonality derived stem cell” refers to a stem cell that is generated by the division of a single stem cell and is genetically identical to that stem cell.

The terms “totipotent”, “pluripotent”, “and “multipotent” refer to cells at different stages in development. The term “totipotent stem cell” refers to a cell that can form after the division of a fertilized egg and can form a blastocyst and develop into a complete individual (e.g. mouse Oct-4+ cells). The term “pluripotent cell” refers to a cell that has the potential to develop into any cell type. The term “multipotent stem cell” refers to a cell that is found in mature tissue with the ability to differentiate into at least two or more differentiated descendant cells are formed by the body to replace worn out cells in tissues and organs (e.g. blood cells, etc.).

The term “plasticity” refers to the ability of stem cells from one adult tissue to generate a differentiated and specialized cell type (s) of another tissue.

The term “feeder layer” refers to cells used in co-culture for a desired effect, for example to maintain pluripotent stem cells.

The term “conditioned medium” refers to culture medium that has been in contact with live cells and contains a range of cell-derived molecules (e.g. growth substances, etc.) that when placed in contact with a subsequent batch of cells may enhance the growth or differentiation of subsequent cells. The term “non-conditioned medium” refers to cell medium that has not been in contact with cells. In the absence of a description, media should be understood to mean non-conditioned media.

The term “fibroblast feeder” refers to a feeder layer comprising fibroblasts. The term “fibroblast” refers to a stellate (star-shaped) or spindle-shaped cell with cytoplasmic processes present in connective tissue, capable of forming fibers such as collagen fibers. The term “mouse fibroblast feeder,” and “mEF” refers to a feeder layer comprising mouse fibroblasts, while the term “conditioned medium from mouse fibroblast feeder” refers to culture medium that has been exposed to mouse fibroblast cells.

The term “laminin” and “LAM” refer to an extracellular matrix protein which contains a number of functional domains that allow it to assemble into sheets to act as a cell attachment substrate and allows it to act as a ligand to act as a growth factor (e.g. inducing differentiation, etc.).

The term “POU” is an acronym derived from the names of three mammalian transcription factors, the pituitary-specific Pit-1, the octamer-binding proteins Oct-1 and Oct-2, and the neural Unc-86 from Caenorhabditis elegans. The term “POU transcription factor” refers to a member of a POU gene family that is a DNA-binding protein capable to activate the transcription of genes bearing cis-acting elements containing an octameric sequence called the octamer motif, within their promoter or enhancer region.

The terms “Oct-4 POU transcription factor”, “octamer-binding transcription factor 3”, “Oct-3”, “OCT3”, “Octamer-binding transcription factor 4”, “Oct-4”, “OCT4”, “POU5F1”, “OTF3”, “class V POU factor Oct-3”, and “POU domain, class 5, transcription factor 1”, refer to a POU transcription factor in undifferentiated stem cells.

The term “nanog” refers to a homeobox transcription factor found in undifferentiated stem cells.

The term “tumor rejection antigen”, “TRA”, and “human embryonal carcinoma marker antigen” refer to keratin sulphate-associated antigens. These include proteins detected with monoclonal antibodies for TRA-1-60, TRA-1-81, TRA-1-85, TRA-2-54, TRA-2-49.

The term “stage-specific embryonic antigens”, “SSEA-1”, “SSEA-2”, “SSEA-3”, and “SSEA-4” refer to a carbohydrate antigen whose cell surface expression changes upon differentiation. For example in murine ES cells, undifferentiated murine pluripotent cells express SSEA-1 while differentiation is characterized by the loss of SSEA-1 expression and may be accompanied, in some instances, by the appearance of SSEA-3 and SSEA-4. For example in humans, undifferentiated human EC, ES and EG cells express the antigens SSEA-3, SSEA-4, TRA-1-60 and TRA-1-81 while differentiating human EC and ES cells are characterized by an increase in SSEA-1 expression and a down regulation of SSEA-3 and SSEA-4.

The terms “Activin A”, “ACTA”, “ACTa”, and “ACT” refer to a homodimer of “inhibin beta-A chain” or “activin beta-A chain”. The term “activin B” refers to a homodimer of “inhibin beta-B chain” or a homodimer of “activin beta-B chain”. The term “activin AB” refers to a dimer of “inhibin beta-A chain and beta-B chain. All of these proteins are members of the Activin protein group of proteins, which are members of the TGFβ family of proteins. The terms “inhibin” and “inhibins” refer to a member of the TGFβ family that inhibits diverse biological functions such as hypothalamic and pituitary hormone secretion, gonadal hormone secretion, stem cell development and maturation, erythroid differentiation, insulin secretion, nerve cell survival, embryonic axial development, bone growth, etc. and may oppose the functions of the activins, wherein their function is related to their subunit composition. The term inhibin includes both inhibin A and Inhibin B.

The terms “keratinocyte growth factor”, “KGF”, “Fibroblast growth factor-7”, “FGF-7”, “HBGF-7”, and “heparin-binding growth factor-7”, refer to a growth factor of the fibroblast growth factor family active on keratinocytes. The term “keratinocyte” refers to a cell that makes keratin. The term “keratin” refers to large molecules found in specialized epithelial cells such as those of the upper layer of the skin, hair, nails, and animal horns.

The term “leukemia inhibitory factor”, “LIF”, “leukemia inhibitory factor precursor”, “differentiation-stimulating factor”, “D factor”, “melanoma-derived LPL inhibitor”, “MLPLI”, “HILDA”, “human interleukin for DA cells”, and “myeloid growth factor human interleukin for DA cells” refer to a cell messenger protein that inhibits differentiation of stem cells and induces terminal differentiation in leukemic cells and induces hematopoietic differentiation in normal and myeloid leukemia cells.

The term “embryo” refers to a developing organism from the time of fertilization until significant differentiation has occurred, for example in humans until the end of the eighth week of gestation, when it becomes known as a fetus.

The terms “STAT” and “Signal Transducers and Activators of Transcription” refer to a molecule in the family of proteins that regulates genes (e.g. STAT1, STAT2, STAT3, STAT4, STAT5. STAT6, etc.). The term “STAT3” refers to an oncogene involved in activating expression of cyclin D1, c-Myc, bcl-xl, etc., and involved in promoting cell-cycle progression, cellular transformation, and in preventing apoptosis.

As used herein, the term “protein kinase” refers to a protein that catalyzes the addition of a phosphate group from a nucleoside triphosphate to an amino acid in a protein. Kinases comprise the largest known enzyme superfamily and vary widely in their target proteins. Kinases can be categorized as protein tyrosine kinases (PTKs), which phosphorylate tyrosine residues (e.g. Janus family tyrosine kinases (JAK)), and protein serine/threonine kinases (STKs), which phosphorylate serine and/or threonine residues and the like.

As used herein, the term “protein phosphatase” refers to proteins that remove a phosphate group from a protein. Protein phosphatases are generally divided into two groups, receptor-type and non-receptor type (e.g. intracellular) proteins. An additional group includes dual specificity phosphatases. Examples of protein phosphatases include, but are not limited to, human protein phosphatase (PROPHO), FIN13, cdc25 tyrosine phosphatase, protein tyrosine phosphatase (PTP) 20, PTP 1D, PTP-D1, PTP .lambda., PTP-S31 (See e.g., U.S. Pat. Nos. 5,853,997; 5,976,853; 5,294,538; 6,004,791; 5,589,375; 5,955,592; 5,958,719; and 5,952,212; all of which are incorporated herein by reference). Examples of targets for protein phosphatase are STATs.

As used herein, a “subject” is any living organism into which stem cells or a substance produced using stem cells can be introduced, including, but not limited to animals. As used herein, the term “animal” includes humans unless otherwise noted by reference to “non-human animals”.

### DESCRIPTION OF EXEMPLARY EMBODIMENTS

The present invention relates to maintenance of the undifferentiated state and/or pluripotency in stem cells. In specific embodiments, it relates to maintaining undifferentiated stem cells, such as human embryonic stem cell lines, using culture medium enriched with a TGFβ family member, such as Activin A, an FGF family member, such as keratinocyte growth factor, nicotinamide (NIC), or a combination of two or all of these, without using fibroblast feeder layers or leukemia inhibitory factor.

In a first aspect, the present invention provides a method for maintenance of undifferentiated stem cells, where the method comprises exposing a stem cell to one or more proteins from the TGFβ family of proteins in an amount sufficient to maintain the cell in an undifferentiated state for a sufficient amount of time to achieve a desired result. In some embodiments, the TGFβ family member is an Activin, such as Activin A. In some embodiments, the FGF family member is KGF. In embodiments, the method is also a method for growth of undifferentiated stem cells. The method can comprise repeating the exposing step with additional (i.e., new) TGFβ family member protein, FGF family member, and/or NIC at the same or a different amount used in the initial exposing. The method is preferably an in vitro method.

As discussed above, the stem cell can be a single cell or population of cells. Furthermore, the cell can be from any species, including human, mouse, rat, monkey, dog, cat, horse, sheep, pig, etc. Likewise, the stem cell can be natural or recombinant. In certain embodiments, fresh biopsied materials are employed to provide stem cells. In other embodiments, cultured stem cells are employed. With regard to the latter, it is not intended that the present invention be limited by the particular culturing method of culturing materials. In one embodiment, the stem cells used in the method are cultured in serum-free culture medium. In one embodiment, the stem cells used in the method are cultured in DSR medium with supplementation (as described in Example 1).

In certain embodiments, the invention provides a method wherein the cells of interest are mammalian. In another embodiment, the invention provides a method wherein the cells of interest are human. A variety of stem cell types can be cultured by the compositions and methods of the present invention, including but not limited to stem cells selected from the group consisting of embryonic and fetal stem cells. It is not intended that the present invention be limited to the sources of stem cells either. Thus, in embodiments, stem cells are derived from embryonic tissue. In other embodiments, stem cells are derived from fetal tissue. In one embodiment, stem cells are derived from blood.

As discussed above, the method does not limit the species from which the stem cell originates. A variety of species may be used as sources of stem cells, including but not limited to human, great apes, monkeys, cows, horses, sheep, pigs, goats, dogs, cats, guinea pigs, rats, mice, goldfish, xenopus, zebrafish, etc. In addition, the invention is not limited in the differentiation state of the cells when exposed to the various substances disclosed herein. Thus, in embodiments, the invention provides a method wherein the cells of interest are undifferentiated. In other embodiments, the invention provides a method wherein the cells of interest are differentiated. In another embodiment, the invention provides a method wherein the contacted cells are undifferentiated. In another embodiment, the invention provides a method wherein the contacted cells are pluripotent. In another embodiment, the invention provides a method wherein the contacted cells are pluripotent in vivo. In another embodiment, the invention provides a method wherein the contacted cells are pluripotent in vitro. In another embodiment, the invention provides a method wherein the contacted cells have the same karyotype as the cells of interest.

According to the method of the invention, exposing can be any action that permits a TGFβ family protein, such as Activin A, to contact a stem cell. Thus, exposing can be simply adding a TGFβ family protein, such as Activin A, to a medium in which a stem cell is present. It also can be adding a precursor of the TGFβ family protein, such as Activin A, to the medium along with a cell, protein, or chemical that can convert the precursor to a functional protein, such as Activin A. Exposing can be performed manually (e.g., by a human adding Activin A to a culture of stem cells) or automatically (e.g., by a machine).

The amount of TGFβ family protein to be used and exposed to the cell may vary depending on the cell type, the desired result, the number of cells, the volume of the media in which the cell is growing, and the speed with which the result is desired to be obtained. Although variation in the amount to be added is to be expected based on these and other parameters, the invention contemplates the use of about 5 ng/ml to about 500 ng/ml of TGFβ family member protein (final concentration in the medium). Thus, in embodiments, the method comprises adding at least one TGFβ family member protein in an amount sufficient to achieve a final concentration of 5 ng/ml, 6 ng/ml, 7 ng/ml, 9 ng/ml, 10 ng/ml, 12 ng/ml, 14 ng/ml, 17 ng/ml, 20 ng/ml, 24 ng/ml, 26 ng/ml, 30 ng/ml, 35 ng/ml, 40 ng/ml, 45 ng/ml, 50 ng/ml, 55 ng/ml, 60 ng/ml, 70 ng/ml, 85 ng/ml, 100 ng/ml, 120 ng/ml, 140 ng/ml, 170 ng/ml, 200 ng/ml, 240 ng/ml, 290 ng/ml, 350 ng/ml, 400 ng/ml, 450 ng/ml, or 500 ng/ml, or any other specific amount between 5 and 500 ng/ml. In embodiments, the TGFβ family member protein is Activin A.

Alternatively, the TGFβ family member protein can be present in the medium in a concentration of from about 0.01 nM to about 100 nM. Thus, in various embodiments, the medium comprises exactly or about 0.01 nM, 0.05 nM, 0.1 nM, 0.5 nM, 1 nM, 2 nM, 3 nM, 4 nM, 5 mM, 8 nM, 10 nM, 16 nM, 25 nM, 32 nM, 45 nM, 64 nM, 75 nM, 85 nm, or 100 nM (or any other specific amount between 0.01 nM and 100 nM) of a TGFβ family member protein, such as Activin A.

The amount of time that the cells are maintained in an undifferentiated state will vary depending on the ultimate desired result. It can vary from one hour or less to days, weeks, or years. For example, the amount of time can be about 10 weeks or more, about 15 weeks or more, about 20 weeks or more, about 25 weeks or more, or about 30 weeks or more. The method permits the practitioner to control stem cell maintenance and growth by adjusting the amount of TGFβ family member protein exposed to the cells, the time of exposure, and the number of times exposure is repeated. It is contemplated that exposures subsequent to the first exposure are to be performed, at least in part, with additional TGFβ family member. According to the invention, subsequent exposures with a TGFβ family member that comprises solely the same TGFβ family member that was previously exposed to the cell is considered merely an extension of the original exposure. The amount of time is conveniently expressed in the number of passages of cells grown in culture. In general, the method permits maintenance or growth of a cell for less than one passage to 30 or more passages. Thus, in particular embodiments, the method permits maintenance or growth of a cell for one passage, two passages, three passages, four passages, five passages, six passages, seven passages, eight passages, nine passages, ten passage, eleven passages, twelve passages, thirteen passages, fourteen passages, fifteen passages, sixteen passages, seventeen passages, eighteen passages, nineteen passages, twenty passages, twenty-one passages, twenty-two passages, twenty-three passages, twenty-four passages, twenty-five passages, twenty-six passages, twenty-seven passages, twenty-eight passages, twenty-nine passages, thirty passages, or more. In addition, according to the invention, exposure of a cell to a TGFβ family member, such as Activin A, permits maintenance or growth of the cell for a fraction of a passage, such as 1/10 of a passage, ⅕ of a passage, ⅓ of a passage, ½ of a passage, ⅔ of a passage, and ⅘ of a passage. In effect, the method can permit a practitioner to maintain and/or grow a stem cell for days, weeks, months, or years.

In view of the standard practice of splitting and saving cell culture cells for future use, the invention contemplates storing the cells that have been treated according to the present methods. Storing can be accomplished by any known technique, including those that involve freezing of cells for long-term storage.

The desired result to be obtained through maintenance and/or growth of the cell can be any medically or scientifically relevant result, such as, but not limited to, production of a particular substance, confluency of the culture on a culture plate, production of a sufficient number of cells for transfer to new culture media (i.e., for passage), or production of a sufficient number of cells for implantation into a subject. There are numerous and varied uses for stem cells that have been proposed and implemented, including, but not limited to repopulation of blood cells after cancer treatments, treatment for CNS degenerative diseases, such as Alzheimer's disease and Parkinson's disease, and treatment for diabetes. Any and all of the proposed and implemented uses are suitable desired results according to the present method.

The TGFβ protein can be naturally obtained or recombinant. Furthermore, as used herein with regard to the method of the invention, the term Activin A includes fragments and derivatives of Activin A, as defined below and above. The sequence of one particular human Activin A encompassed by the term Activin A is given in SEQ ID NO:1. Other non-limiting examples of Activin A are given in SEQ ID NO:2-16, while non-limiting examples of nucleic acids encoding Activin A are given in SEQ ID NO:33-34.

The method can comprise exposing the stem cell to a member of the FGF family of proteins, such as KGF, in an amount sufficient to permit maintenance and/or growth of the cell for a sufficient amount of time to achieve a desired result. It has been found that addition of an FGF family member, such as KGF, to cultures of stem cells improves growth of those cells. In particular, addition of an FGF family member stops the slowing and cessation of growth that is typically seen in stem cell cultures. Indeed, it has been found that addition of an FGF family member, such as KGF, to a stem cell culture permits maintenance and growth of the culture for multiple passages, including, but not necessarily limited to 10 or more passages. This effect is particularly pronounced in cultures also containing a TGFβ family member protein, such as Activin A.

In embodiments where the FGF family member protein is exposed to cells along with a TGFβ family member protein, the FGF family member protein can be exposed to the cell before, at the same time, or after exposure to the TGFβ family member protein.

As the FGF family member protein is being exposed to the same cells as the TGFβ family member protein, it is evident that the disclosure above with regard to cells is equally applicable to methods that comprise exposing cells to an FGF family member protein. Likewise, the results that can be desired will overlap with those disclosed above and will be any desired by those of skill in the art.

The FGF family member protein can be exposed to the cells in any amount that is sufficient to achieve a desired result, in accordance with the discussion above. Exposure can be accomplished by any of the actions discussed above with respect to the TGFβ family member protein. Among the desired results contemplated, a particularly interesting one is growth of stem cells for a relatively long time, such as for 10 or more passages. Accordingly, the amount of time can be one passage, two passages, three passages, four passages, five passages, six passages, seven passages, eight passages, nine passages, ten passage, or more. In accordance with the discussion above, it can be any number of passages or partial passages up to 30 passages or more. Furthermore, any portion of one or more passages may be a desired amount of time. Thus, the amount of time can range from less than one hour to a day or more to a week or more. The amount of time can be about 5 weeks, about 10 weeks, about 15 weeks, about 20 weeks, about 25 weeks, about 30 weeks, or more, including any specific number of weeks in between these exemplary numbers.

In various embodiments, the FGF family protein is exposed to the cell in such a manner that it is present in the medium comprising the cell in a concentration of 0.5 ng/ml to 1 mg/ml. Thus, in particular embodiments, the FGF family member protein, such as KGF, is present in an amount of exactly or about 0.50 ng/ml, 0.75 ng/ml, 1 ng/ml, 2 ng/ml, 3 ng/ml, 5 ng/ml, 7.5 ng/ml, 10 ng/ml, 20 ng/ml, 30 ng/ml, 40 ng/ml, 45 ng/ml, 50 ng/ml, 55 ng/ml, 60 ng/ml, 75 ng/ml, 85 ng/ml, 100 ng/ml, 125 ng/ml, 150 ng/ml, 175 ng/ml, 200 ng/ml, 250 ng/ml, 350 ng/ml, or 500 ng/ml (or any amount between 0.50 ng/ml, and 1 mg/ml).

As with exposing the cell to a TGFβ family member protein, exposing the cell to an FGF family member protein can comprise repeating the exposing step. Repetition of exposing an FGF family member protein to the cell is contemplated under the same parameters discussed above with regard exposing a TGFβ family member protein, such as Activin A, to the cell.

The FGF family member protein can be naturally obtained or recombinant. Furthermore, as used herein with regard to particular embodiments of the method of the invention, the term KGF includes fragments and derivatives of KGF as well as molecules considered as species of KGF, as defined below and above. The sequence of one particular human KGF encompassed by the term KGF is given in SEQ ID NO:17. Other non-limiting examples of KGF are given in SEQ ID NO:18-24, while non-limiting examples of nucleic acids encoding KGF are given in SEQ ID NO:25-31.

The method of the invention can likewise comprise exposing stem cells to nicotinamide (NIC) in an amount sufficient to permit growth and maintenance of the cells in culture through multiple passages. It has been found that culturing stem cells in NIC permits maintenance and growth for an extended period of time. Thus, according to this embodiment of the method, the desired result can be passage of the cell through at least one, five, nine, ten, eleven, twelve, thirteen, fourteen, fifteen, sixteen, seventeen, eighteen, nineteen, twenty, or more passages (including any other whole number of passages within this range, and fractions thereof).

Exposing a cell to NIC can be accomplished using the actions and considerations discussed above with regard to a TGFβ family protein and/or an FGF family protein. Likewise, in addition to extending the maintenance and growth period of the cells, NIC may be exposed to the cell for any of a number of reasons that would be apparent to those of skill in the art, including those discussed above with regard to TGFβ family member proteins. The cells and characteristics listed above are equally applicable to embodiments comprising exposing the cells to NIC, as are the results and the considerations with regard to repeating exposure.

As with the TGFβ and FGF family members, the amount of NIC to be exposed to the cell can vary depending on the desired result, the amount of cells, and other parameters. In general, amounts of NIC that can be present in the medium containing the cell can vary from about 0.5 mM to about 500 mM. Thus, in particular embodiments, NIC is present in the medium in a concentration of exactly or about 0.5 mM, 1 mM, 1.5 mM, 2 mM, 3 mM, 4 mM, 5 mM, 6 mM, 7 mM, 8 mM, 9 mM, 10 mM, 11 mM, 12 mM, 13 mM, 14 mM, 15 mM, 17 mM, 20 mM, 24 mM, 26 mM, 30 mM, 35 mM, 40 mM, 45 mM, 50 mM, 60 mM, 70 mM, 85 mM, 100 mM, 120 mM, 140 mM, 170 mM, 200 mM, 240 mM, 290 mM, 350 mM, 400 mM, 450 mM, or 500 mM.

As discussed above, exposure of cells to NIC permits extended periods of growth with no change in pluripotent status or karyotype. In addition, certain well-known surface markers remain on the cell surface after extended exposure to NIC.

In general, the methods of the present invention can permit growth and maintenance of stem cells in culture in the absence of any feeder cells, conditioned media, and/or leukemia inhibitory factor (LIF). In embodiments, the methods maintain the cells in a pluripotent state. In various embodiments, the method is a method of inhibiting or delaying differentiation of undifferentiated stem cells. In embodiments, it is a method of inhibiting or delaying loss of pluripotency of stem cells (i.e., delaying commitment of a stem cell to a particular cell type).

In view of the above disclosure, it is evident that the invention provides a method for maintaining and/or proliferating mammalian embryonic stem (ES) cells, where the method comprises: a) providing i) ES cells of interest, and ii) one or more of Activin A, keratinocyte growth factor, and nicotinamide; and b) contacting the ES cells of interest with the Activin A, keratinocyte growth factor, and/or nicotinamide to produce contacted ES cells. Among other things, such a method provides a means to maintain undifferentiated stem cells, a means to promote cell proliferation through cell division, and a means to maintain cells in an undifferentiated state while promoting proliferation in order to increase numbers of undifferentiated cells. In embodiments, this method is a method wherein the contacting is in the absence of mouse fibroblast feeder cells. In yet other embodiments, the invention is a method wherein the contacting is in the absence of conditioned medium from mouse fibroblast feeder cells. In further embodiments, the invention is a method wherein the contacting is in the absence of leukemia inhibitory factor. In addition, in embodiments where maintenance is achieved and embodiments where the cells remain in contact with the Activin A, KGF, and/or NIC for a period of time sufficient to permit growth of the cells, the contact is performed, at least partially, in the absence of mouse fibroblast feeder cells, conditioned medium from mouse fibroblast feeder cells, and/or leukemia inhibitory factor. It is not intended that the present invention be limited to the manner in which the stem cells are maintained and grown.

In a particular embodiment, the invention provides a method for maintaining and/or proliferating mammalian embryonic stem (ES) cells, wherein the method comprises: a) providing; i) ES cells of interest, and ii) one or more of a first polypeptide having at least 90% identity to SEQ ID NO:1 and/or a second polypeptide having at least 90% homology to SEQ ID NO:17; and b) contacting the ES cells in vitro with the first and second polypeptides to produce contacted ES cells. In embodiments, the invention provides a method wherein the concentration of the first polypeptide maintains the contacted ES cells in an undifferentiated state. In another embodiment, the invention provides a method wherein the concentration of the second polypeptide maintains proliferation of said contacted ES cells.

It is to be noted that stem cells treated in accordance with the methods of this invention typically show normal karyotype and markers for those undifferentiated cells, and typically remain pluripotent.

In a second aspect of the invention, the invention provides compositions that can be used to maintain and/or grow stem cells in an undifferentiated or pluripotent state, or to compositions comprising the stem cells. Generally, the compositions comprise a stem cell and/or a TGFβ family member protein, such as Activin A, an FGF family member protein, such as KGF, and/or NIC in an amount and form that is sufficient to permit growth and/or maintenance of at least one culture of stem cells in an undifferentiated state for a sufficient amount of time to achieve a desired result. In accordance with the discussion of the methods, above, the desired result can be any medically or scientifically relevant result, including preparing sufficient numbers of cells for storage. Thus, compositions according to this aspect of the invention are those that are useful in practicing the method of the present invention. In embodiments, the FGF family member protein is not basic fibroblast growth factor (bFGF). In embodiments, the composition is a pharmaceutical composition comprising a stem cell, a TGFβ family member, an FGF family member, NIC, or a combination of two or more of these. The compositions can be useful for, among other things, stem cell therapeutics.

Compositions comprising a TGFβ family member protein may comprise any form of the member protein, from any source. Accordingly, compositions comprising Activin A may comprise any form of the protein, from any source. As discussed above, numerous examples of Activin A proteins are given in the Sequence Listing and listed herein by GenBank accession numbers. Others are encompassed as well. Likewise, derivatives, such as those having a sequence presented in the Sequence Listing or known in the art, but having post-translational modifications that do not negatively affect the ability of the Activin A to promote maintenance and growth of stem cells in an undifferentiated state, are encompassed by the present invention. For example, variants having substantial amino acid identity with one or more of the sequences presented herein or known in the art as Activin A sequences are encompassed. Thus, compositions comprising a protein sharing 80%, 90%, 95%, 96%, 97%, 98%, 99%, or more identity with one or more of the sequences disclosed herein, such as SEQ ID NO:1, are encompassed by the present compositions. In embodiments, the invention provides a composition comprising recombinant Activin A, which comprises a polypeptide at least 90% identical to SEQ ID NO:1. Accordingly in other embodiments, recombinant Activin A polypeptides are at least 90%, 95%, 96%, 97%, 98%, 99% (or more) identical to any of SEQ ID NOs:1 or 3-16. In a particular embodiment, the invention provides a composition comprising recombinant human Activin A.

In certain embodiments, the composition can comprise a nucleic acid that encodes a TGFβ family member, such as Activin A. In these embodiments, the composition also comprises other substances that permit expression of the nucleic acid to express the Activin A. In these embodiments, the nucleic acids are those disclosed herein or those known to persons of skill in the art. The nucleic acids also include nucleic acids that hybridize to one or more Activin A-encoding nucleic acids under stringent hybridization conditions, or nucleic acids that share high identity with one or more Activin A-encoding nucleic acids, such as 80%, 90%, 95%, 98%, 99%, or more identity.

Compositions comprising an FGF family member protein may comprise any form of the member protein, from any source. Accordingly, compositions comprising KGF may comprise any form of the protein, from any source. As discussed above, numerous examples of KGF proteins are given in the Sequence Listing. Others are encompassed as well, such as, but not limited to, fibroblast growth factors (FGF). Likewise, derivatives, such as those having a sequence presented in the Sequence Listing, listed above, or known in the art, but having post-translational modifications that do not negatively affect the ability of the FGF family member protein to promote maintenance and growth of stem cells in an undifferentiated state, are encompassed by the present invention. For example, variants having substantial amino acid identity with one or more of the sequences presented herein or known in the art as KGF sequences are encompassed. Thus, compositions comprising a protein sharing 80%, 90%, 95%, 96%, 97%, 98%, 99%, or more identity with one or more of the sequences disclosed herein, such as SEQ ID NO:17, are encompassed by the present compositions.

In certain embodiments, the composition can comprise a nucleic acid that encodes an FGF family member protein, such as KGF. In these embodiments, the composition also comprises other substances that permit expression of the nucleic acid to express the protein. In these embodiments, the nucleic acids are those disclosed herein or those known to persons of skill in the art. The nucleic acids also include nucleic acids that hybridize to one or more FGF family-encoding nucleic acids under stringent hybridization conditions, or nucleic acids that share high identity with one or more FGF family-encoding nucleic acids, such as 80%, 90%, 95%, 98%, 99%, or more identity. As will be understood by those of skill in the art, it may be advantageous to produce a nucleotide sequence encoding a protein of interest, wherein the nucleotide sequence possesses non-naturally occurring codons. Therefore, in some embodiments, codons preferred by a particular prokaryotic or eukaryotic host (Murray et al., Nucl. Acids Res., 17 (1989)) are selected, for example, to increase the rate of expression or to produce recombinant RNA transcripts having desirable properties, such as a longer half-life, than transcripts produced from naturally occurring sequence. The invention does not limit the source (e.g., cell type, tissue, animal, etc.), nature (e.g., synthetic, recombinant, purified from cell extract, etc.), and/or sequence of the nucleotide sequence of interest and/or protein of interest.

In embodiments, the invention provides compositions comprising a TGFβ family member and an FGF family member. In one embodiment, the invention provides a composition comprising Activin A (ACTa) and keratinocyte growth factor (GF), wherein the concentration of ACTa in the composition maintains embryonic stem cells in an undifferentiated state, and the concentration of KGF in the composition maintains proliferation of embryonic stem cells. In one embodiment, the present invention relates to compositions for the maintenance of the undifferentiated state with Activin A, and for increasing proliferation with keratinocyte growth factor, NIC, or both.

In embodiments, the Activin A, KGF, or both are recombinant.

In one embodiment, the invention provides a composition comprising a first polypeptide having at least 90% identity to SEQ ID NO:1, and a second polypeptide having at least 90% identity to SEQ ID NO:17, wherein the concentration of the first polypeptide in the composition is sufficient to maintain embryonic stem cells in an undifferentiated state, and the concentration of the second polypeptide in the composition is sufficient to maintain proliferation of embryonic stem cells. Accordingly, in other embodiments, the first polypeptide is at least 90%, 95%, 96%, 97%, 98%, 99% (or more) identical to any of SEQ ID NOs:1 and 3-16. Likewise, in other embodiments, the second polypeptide is at least 90%, 95%, 96%, 97%, 98%, 99% (or more) identical to any of SEQ ID NOs:17-24.

In one embodiment, a variant of Activin A and/or KGF is present, and the sequence of each variant independently has at least 95% identity, at least 90% identity, at least 85% identity, at least 80% identity, at least 75% identity, at least 70% identity, and/or at least 65% identity with the sequence of one of the sequences disclosed herein.

In another embodiment, the invention provides a composition comprising a recombinant KGF, wherein the recombinant keratinocyte growth factor comprises a polypeptide at least 90% identical to SEQ ID NO:17. In another embodiment, the invention provides a composition in which the keratinocyte growth factor is from one or more of SEQ ID NOs:11-14. Accordingly in other embodiments, the second polypeptide is at least 90%, 95%, 96%, 97%, 98%, 99% (or more) identical to any of SEQ ID NOs: 17-24.

A composition according to the invention can comprise nicotinamide. NIC can be present in the composition in an amount sufficient to permit at least one culture of stem cells to maintain viability and/or grow for a desired length of time.

The TGFβ and FGF family members and the NIC can independently be provided in any physical form, including in liquid or solid (dried) form. When provided in liquid form, additional stabilizers may be included to aid in storage. Dried forms can include additional substances that are included during the drying, which may be added to aid in stability during drying or rehydration.

The compositions of the invention generally comprise a component in addition to TGFβ and FGF family members, and the NIC. The additional component can be any substance that is known to be suitable for, or compatible with, growth of stem cells or for introduction of stem cells or substances produced by stem cells into an animal or human subject. For example, it may include water or an aqueous solution containing, for example, salts (e.g., NaCl), detergents (e.g., SDS), and other components (e.g., Denhardt's solution, dry milk, salmon sperm DNA, etc.). Alternatively, it may contain blood or blood products, such as serum or serum components. Thus, it may be growth (i.e., culture) media, including growth media generally known in the art as “KO” serum media or media containing “KO” serum. The culture medium can comprise a TGFβ family member, an FGF family member, NIC, or a combination of two or all three of these. While each and every component of the composition need not be biologically compatible, it is preferred that the components either be biologically compatible or be present in a concentration that is sufficiently low as to minimize the negative biological effects of the component to a level that is acceptable, based on any number of criteria typically used to evaluate biocompatibility. Preferably, the composition comprising TGFβ and FGF family members, and the NIC is sterile (except, of course, with regard to stem cells or other cells intentionally included in the composition). In view of the usefulness of the compositions of the invention in growing and maintaining stem cells, it is evident that the compositions of the invention can themselves comprise stem cells, including embryonic stem cells, such as hESC.

As mentioned immediately above, in certain embodiments, the composition comprises stem cells. The stem cells can be cells grown in the presence of one or more TGFβ family members, one or more FGF family members, NIC, or a combination of two or all three of these. The stem cells may be, but are not necessarily, in an undifferentiated state. Furthermore, while it is often desirable to have all of the cells in a composition in an undifferentiated state, it is recognized that, due to the heterogeneity in growth and survival of cells, it is likely that less than 100% of the cells in the composition will have the characteristics desired. This fact is not to be understood as excluding compositions having a significant number (e.g., 50%, 60%, 70%, 80%, 90%, 95%, 97%, 98%, 99%, or more) of cells in an undifferentiated or pluripotent state.

In a third aspect, the invention provides kits that contain some or all of the materials that are needed to grow and/or maintain embryonic stem cells in an undifferentiated or pluripotent state. In its most basic form, the kit comprises at least one container containing a TGFβ family member, such as Activin A, an FGF family member, such as KGF, NIC, or a combination of two or all three of these in an amount sufficient to grow and maintain stem cells in culture for a sufficient amount of time to achieve a desired result. Preferably, the cells are in an undifferentiated, pluripotent state. The desired result can be any medically or scientifically relevant result, such as production of a detectable amount of a particular substance, confluency of the culture on a culture plate, production of a sufficient number of cells for implantation into a subject, and the like. In embodiments, the kit includes all of the components needed to practice one or more embodiment of the methods of the invention. Ancillary components are also included in certain embodiments of the kit, as are instructions for using the materials in the kit in accordance with the invention.

Thus, in certain embodiments, the kit comprises a single container containing a TGFβ family member (such as Activin A), an FGF family member (such as KGF), NIC, or a combination of two or all three of these. In other embodiments, the kit comprises two or more containers, each containing one or more of a TGFβ family member, such as Activin A, an FGF family member, such as KGF, and NIC. In the latter case, the containers are provided separately in a packaged combination (i.e., in a single package, which can be, but is not necessarily, the kit itself). That is, a composition comprising, for example, Activin A, KGF, NIC, or a combination of two or all three of these is contained in a first container, and another composition independently comprising one or more of these components (in any amounts) is contained in a second, different container, both being included in a single package. Multiple containers of various compositions according to the invention (as well as other compositions useful in the invention) can be included in a single kit to permit the practitioner to make various volumes of materials to be used, for example, in practicing the methods of the invention. In this way, practitioners may practice the invention multiple times or on large volumes of cells without having to obtain multiple kits.

The amount of TGFβ and FGF family member proteins and/or NIC are independently selected to be sufficient to permit at least one culture of stem cells to be maintained or grown for a desired amount of time. Each substance can be contained in its own container or two or more can be contained in a single container. In this way, the practitioner can select the appropriate substance or combination of substances for the specific application needed.

In certain embodiments, at least two of a TGFβ family member protein, such as Activin A, an FGF family member protein, such as KGF, and NIC are provided together in a packaged combination. That is, at least two of these, and preferably three, are contained in a single container within the kit. Providing the two or three together in the kit eliminates the need for the practitioner to combine them after opening the kit, and can reduce the time needed to practice the methods of the invention, or minimize errors in measuring the components or loss of materials due to spills, etc. As with other embodiments, kits according to this embodiment can include multiple containers within the kit, each container containing the same materials, different materials, or a combination where some containers contain the same material as others, while some containers contain unique contents.

In embodiments, multiple (e.g., two, three, four, five, six) containers of one or more individual components (e.g., Activin A, KGF, and NIC) are included in the kit. In embodiments, multiple (e.g., two, three, four, five, six) containers of two or more components together are included in the kit. In embodiments, gloves and/or other supplies or reagents (including, but not limited to, sterile water or a sterile aqueous solution for rehydrating one or more component of the kit) are also included.

As can be seen from the above discussion, where desired, additional components can be included in the kits other than the TGFβ family member protein, the FGF family member protein, and/or NIC. For example, solvents or diluents for these substances can be included. Furthermore, materials for measuring or delivering the components of the kit can be included, such as syringes, pipettes, and the like. Each of these additional components, etc. can be included in packages of one or more item, or can be included as separate, unpackaged items.

A container can be anything that is suitable for containing the TGFβ family protein, FGF family protein, and/or NIC, and/or optional additional components to be contained. Thus, containers can be, but are not limited to, tubes, ampoules, vials, cans, bags, or jars, such as those made of metal, plastic, rubber, saran, and glass. In embodiments, the container can be the delivery device, such as a syringe or pipette suitable for supplying the component to a media comprising the stem cells. The containers are preferably re-sealable or automatically sealing to preserve unused contents after initial opening. The containers and their contents are preferably sterile (or sterilized prior to opening).

The kits themselves can be fabricated from any suitable material, such as cardboard, plastic, metal, or glass. Cardboard and plastic are preferred materials for the kits.

Instructions for using one or more components of the kit, or for practicing the methods of the invention, may be included in the kit. The instructions may be provided as a separate component, such as printed material on a paper, card, plastic sheet, or the like. Alternatively, the instructions may be provided on the kit itself, for example, on a side or the top or bottom of the kit. Alternatively, the instructions may be provided on a container for a component of the kit.

In a fourth aspect, the present invention provides stem cells that are undifferentiated and pluripotent. The stem cells can be provided in compositions that do not comprise feeder cells, conditioned media from mouse embryonic feeder layers, and/or STAT3 activation. In embodiments, the stem cells are provided in a composition that comprises a TGFβ family member protein, such as Activin A, an FGF family member protein, such as KGF, NIC, or a combination of two or more of these. In embodiments, the composition comprises Activin A, KGF, and NIC. Although the stem cells can be any cell disclosed above, in preferred embodiments, the cells are human stem cells, such as human embryonic stem cells.

In general, the stem cells are cells produced through use of the methods of the invention. That is, stem cells of the invention are those produced by exposure of a stem cell to a TGFβ family member protein, an FGF family member protein, NIC, or a combination of two or all three of these. The stem cells are exposed to one or more of these substances in the absence of conditioned medium, feeder cells, or LIF. The stem cells can be cells that have been passaged multiple times, as described above.

### EXAMPLES

The invention will be further explained by the following Example, which is intended to be purely exemplary of the invention, and should not be considered as limiting the invention in any way.

### Example 1

### Maintenance of Pluripotency of Stem Cells in the Absence of Feeder Cells or Conditioned Media

The present invention relates to compositions and methods for maintenance of the undifferentiated state and/or pluripotency in embryonic stem (ES) cells, and in particular, maintaining undifferentiated human embryonic stem cell lines, using culture medium enriched with a TGFβ family member, an FGF family member, and/or NIC. This Example shows that growth in the presence of the TGFβ family member Activin A maintains pluripotency of stem cells, and that addition of the FGF family member keratinocyte growth factor (KGF) lengthens the amount of time that cells can be grown without entering senescence. It also shows that addition of NIC to culture media lengthens the amount of time that cells can be grown without senescence.

Maintenance of the undifferentiated state and pluripotency in mouse ES requires the presence of mouse fibroblast feeder layers (mEFs) or activation of STAT3 with leukemia inhibitory factor (LIF).¹ Human embryonic stem cell lines (hES) have only recently become available for research,^{2, 3} and the intracellular pathways for self renewal and differentiation are, at this time, largely unknown. Recently the present inventors and colleagues⁴ and others⁵ have shown that STAT3 activation is not sufficient to block differentiation of human ES cell lines when grown on mEFs or when treated with conditioned media from mEFs. The present Example shows that culture medium enriched with Activin A is capable of maintaining hES in the undifferentiated state for at least 10 passages, without the need for feeder layers, conditioned medium from mEFs, or STAT3 activation. hES cells retained markers of undifferentiated cells, including OCT-4, nanog and TRA-1-60, and remained pluripotent, as shown by the in vivo formation of teratomas.

It has been published that pluripotentiality of hES can only be maintained when grown on mEFs,^{2, 3} conditioned medium from mEFs,⁶ or on human feeder layers.⁷ In addition, it has been reported that the signals received from the feeder layers do not operate through the LIF/gp130 pathway.^{4, 5} Therefore alternative pathways, likely triggered by the contact of hES cells to feeder layers and/or soluble factor(s) present in the conditioned media (CM), must be responsible for maintenance of pluripotency. As the inventors and their colleagues have previously shown,⁴ under phase contrast microscope and using histological staining, growth and phenotypic characteristics of HSF6 are similar on feeder layers and in mEF CM when grown on laminin coated dishes. In this case, the cells grow in distinct undifferentiated colonies. Thus, soluble factors secreted by the feeder layers appear to be instrumental in maintaining pluripotency. To determine what these factors might be, we tested various combinations of growth factors, based on our experience in culturing human fetal pancreatic tissue. The inventors used laminin 1 for adhesion, based on preliminary experiments, and the high levels of a6 b1 expression in hES cells.⁶ However, it is to be understood that other known substrates for adhesion, such as fibronectin and collagen, could be used equally effectively.

Here it is shown that when hES cells were grown on laminin in the presence of Activin A and KGF they remained undifferentiated following continuous growth over 10 passages, staining uniformly for the stem cell markers TRA-1-60 and OCT-4, comparable to the staining for cells on feeder layers or in CM. Robust gene expression of oct-4, nanog, and telomerase was also observed by RT-PCR in the cell monolayers, with levels comparable to those obtained in colonies growing on feeder layers. Morphology gradually changed from the usual tight colony formation, to an irregular monolayer of uniformly shaped cells, that appeared larger than what is seen in the original colonies. If allowed, cells eventually formed a continuous monolayer and mounded up in the dish. However these morphologic changes were reversible; when cells were placed back on feeder layers they gradually resumed the colony formation similar to the expected morphology on feeder layers.

When Activin A was removed from the growth medium, the cell morphology rapidly changed to a more differentiated type; after 1 week the cells no longer expressed nanog, with concomitant loss of immunoreactive TRA-1-60 and reduced levels of OCT-4 protein. When Activin A was replaced with BMP-4, the cells were unable to maintain their undifferentiated phenotype, with loss of expression for nanog, oct-4, and telomerase after 1 week; when KGF was removed, the cells maintained their undifferentiated phenotype but the proliferation rate decreased and they could not be subcultured beyond one passage.

Activin A, a member of the transforming growth factor β family, was initially isolated from porcine follicular fluid^{8, 9} as a stimulator of FSH synthesis and secretion. “Activin”, “activin A”, “activin B”, “activin AB”, “erythroid differentiation protein”, and “EDF” are members of the TGF-beta-family, which activates diverse biological functions such as hypothalamic and pituitary hormone secretion, gonadal hormone secretion, stem cell development and maturation, erythroid differentiation, insulin secretion, nerve cell survival, embryonic axial development, bone growth, etc., wherein their function is related to their subunit composition. It has been identified in a wide variety of tissues as an autocrine or paracrine regulator of diverse biological functions (for review see¹⁰). Importantly, the inventors have found high expression of Activin A transcripts in mEFs and abundant secreted protein in the conditioned medium from mEFs. Moreover, the HSF6 cells differentiated when grown on mEFs in the presence of follistatin, a natural inhibitor of Activin A.¹⁰ Interestingly, Activin A has been shown to be secreted by other mesenchymal cells, and its secretion is upregulated by FGF2,¹¹ which is used routinely when culturing hES on mEFs. Taken together these data imply that Activin A, secreted by the mEFs is responsible for maintenance of “stemness” in hES cells.

The inhibition of differentiation in hES was specific for Activin A. While BMP-4, another TGF β family member can maintain pluripotency in mouse ES cells,¹² this is not the case with hES. This is not the only difference between these two cells—mES and hES also differ in their dependence on LIF for maintenance.⁴ In mES cells, BMP-4 plays a paradoxical role in both maintenance of pluripotency and differentiation,^{12, 13} most likely depending on other factors present or on stage of development. hES cells differentiated rapidly in the presence of BMP-4 and KGF, and expression of oct-4, nanog, and telomerase was lost after 1 week culture.

Activin A has also been implicated in differentiation of mES into mesoderm,¹³ of human pancreatic precursor cells into β cells,¹⁴ inhibition of neural differentiation,^{15, 16} and more recently induction of endoderm in hES cells.¹⁷ The present disclosure, however, is the first documentation of an important role for Activin A in maintenance of stem cells in the undifferentiated state, and of its presence in conditioned medium from mEFs.

Teratomas grown in nude mice after transplantation of hES grown in monolayer in the presence of Activin A and KGF showed many ectodermal, endodermal, and mesodermal structures. In addition, RT-PCR performed on RNA from embryoid bodies showed gene expression specific for all 3 embryonic cell layers. These data show that maintenance of hES in medium containing Activin A allows the maintenance of pluripotentiality without the need for co-culture with other foreign or human cells.

Non-transmembrane PTKs form signaling complexes with the cytosolic domains of plasma membrane receptors. Receptors that signal through non-transmembrane PTKs include cytokine (e.g. glycoprotein 130 (gp130) family), hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity.²² Regulation of PTK activity may therefore be an important strategy in controlling some types of cancer.

Materials and Methods: The following is a description of exemplary materials and methods that were used in the Example.

Stem cell culture: hES cell line HSF6 was maintained on mitomycin C treated CF1 mouse feeder layers (mEF) at 37° C., 5% CO₂ in DSR medium, which consisted of high glucose DMEM containing knockout serum replacer, glutamine, non-essential amino acids, 0.1 mM β-mercaptoethanol (all from Gibco, Carlsbad, Calif.; www.invitrogen.com) as previously described,¹⁹ or on laminin (20 μg/ml, Chemicon, www.chemicon.com) coated dishes or in DSR containing 50 ng/ml human recombinant Activin A (ACT A), 50 ng/ml human recombinant Keratinocyte Growth Factor (KGF), both from Preprotech Inc. (Rocky Hill N.J.; www.preprotech.com). These concentrations were determined from previous experiments with human fetal pancreatic cell culture.^{14, 20} In some experiments, 10 ng/ml human recombinant Bone Morphogenetic Protein 4 (BMP-4; R&D Systems Minneapolis, Minn. www.RnDSystems.com) was used to replace ACT A, and 0.4 μg/ml follistatin (R&D Systems) was added to ES cells grown on mEFs, sufficient to neutralize 50 ng/ml Act A according to the manufacturer's directions. Medium was changed every day on cells grown on mEFs and every other day, on cells grown on laminin with the growth factors. Cells were passaged weekly at 1:3 or 1:4 dilution.

Alternatively, hES cell line HSF6 was maintained on mitomycin C treated CF1 mouse feeder layers (mEF) at 37° C., 5% CO₂ in DSR medium as previously described⁴. For the experiments described here, passage 43 hES cells were cultured on laminin (20 μg/ml, Chemicon, www.chemicon.com) coated dishes in the presence of conditioned medium from mEFs (CM) supplemented with 10 ng/ml basic fibroblast growth factor (FGF2; Preprotech Inc. Rocky Hill N.J.; www.preprotech.com), or on laminin in DSR medium containing 50 ng/ml human recombinant activin A, 50 ng/ml human recombinant KGF, both from Preprotech, and 10 mM Nicotinamide (NIC; Sigma Corp, St Louis Mo.). A dose response with hES cells using activin A at 5, 50, and 100 ng/ml, showed 50 ng/ml to be optimal to maintain the cells in an undifferentiated state.

In some experiments, 10 ng/ml human recombinant Bone Morphogenetic Protein 4 (BMP-4; R&D Systems Minneapolis, Minn. www.RnDSystems.com) was used to replace activin A, and 2 μg/ml recombinant mouse FS-288 follistatin (R&D Systems) was added to ES cells grown on mEFs, sufficient to neutralize 50 ng/ml activin A according to manufacturer's directions. Medium was changed every day on cells grown on mEFs or in CM and every other day, on cells grown on laminin with the growth factors. Cells were passaged weekly at 1:3 or 1:4 dilution by gentle treatment with 1 mg/ml Collagenase IV (Gibco BRL) for 5 minutes, followed by scraping.

Immunohistochemistry: Stem cell cultures were grown on coverslips coated with mEFs or laminin fixed with 4% paraformaldehyde and stained. Protein expression of the stem cell markers TRA-1-60 and Oct-4 was analyzed by immunohistochemistry using primary mouse anti-TRA-1-60 (Chemicon) and rabbit anti-OCT-4 antiserum (a generous gift from Dr Hans Scholer, U Penn). Control slides were incubated with mouse IgM and rabbit IgG. Affinity purified rhodamine red-conjugated donkey anti-mouse IgM and fluorescein-conjugated donkey anti-rabbit IgG (Jackson Immunoresearch) were directed against primary antibodies. Coverslips were mounted in anti fade medium (Biomeda Corp, Foster City, Calif.; http://biomeda.com) and viewed on a Nikon eclipse E800 microscope (NikonUSA, Melville, N.Y.; www.nikonusa.com) equipped with a fluorescent attachment. Images were captured with a SPOT digital camera (Diagnostic Instruments Inc, Sterling Heights, Mich.; www.diaginc.com) and acquired through Image Pro Plus 4.0 (Media Cybernetics, Silver Spring, Md.; www.mediacy.com). Color composite pictures were processed using Adobe Photoshop 7.0 (Adobe Systems, Mountainview, Calif.; www.adobe.com).

Alternatively, hES cell cultures were grown on coverslips coated with mEFs or laminin, fixed with 4% paraformaldehyde and immunostained as previously described.⁴ Protein expression of the stem cell markers TRA-1-60, SSEA-4 and Oct-4 was analyzed using primary mouse anti-TRA-1-60 IgM (Chemicon) mouse anti-SSEA-4 IgG3 (DSHB, U of Iowa, Iowa City, Iowa), and rabbit anti-Oct-4 antiserum (a generous gift from Dr Hans Scholer, U Penn). Control slides were incubated with mouse IgM or IgG and rabbit IgG.

RT-PCR: RNA was purified using the RNeasy minikit including DNase treatment (Qiagen, Valencia, Calif.; wwwl.qiagen.com) and reverse transcribed using AMV with 3.2 μg of random primer (both Roche, Indianapolis, Ind.; www.roche-applied-science.com) and 1 μg of total RNA in a reaction volume of 20 μL. 1 μL of cDNA was used for each PCR reaction in a total volume of 50 μL. β-actin expression was used for sample quantitation and comparison (i.e., as an internal control).

Probes were prepared specific for human oct-4, nanog, and telomerase. Oligonucleotide primers used in the Examples had the following sequences:

β-actin
forward:
cgcaccactggcattgtcat
reverse:
ttctccttgatgtcacgcac
oct-4
forward:
gagcaaaacccggaggagt
reverse:
ttctctttcgggcctgcac
nanog
forward:
gcftgccttgctttgaagca
reverse:
ffcttgactgggaccffgtc
Activin A
forward:
cttgaagaagagacccgat
reverse:
cttctgcacgctccactac
Neuro-D:
forward:
gagactatcaetgctcagga
reverse:
gataagcccttgcaaagcgt
Brachyury
T gene:
forward:
caaccaccgctggaagtac
reverse:
ccgctatgaactgggtctc
α-feto-
protein:
forward:
agaacctgtcacaagctgtg
reverse:
gacagcaagctgaggatgtc
ALK-4:
forward:
cacgtgtgagacagatggg
reverse:
ggcggttgtgatagacacg
ACVR-2:
forward:
gggagctgctgcaaagttg
reverse:
ccacatcaacactggtgcc
ACVR-2B:
forward:
caccatcgagctcgtgaag
reverse:
gagcccttgtcatggaagg
hTERT
forward:
cagctcccatffcatcagca
reverse:
cgacatccctgcgttcttg

PCR products were loaded onto a 1.2% agarose gel (1.6% gel for htert) and stained with ethidium bromide.

Pluripotency: pluripotency was assessed in vivo by examining teratoma formation 8 weeks after transplanting the hES cells under the renal capsule of nude mice as previously described for analysis of pancreatic progenitor islet cell differentiation.²¹ Briefly, hES were removed from laminin or mEFs, and allowed to form embryoid bodies overnight in Costar Ultra Low Cluster dishes (Corning Inc, Corning N.Y., www.corning.com). They were centrifuged into a pellet, collected in a 10 μl positive pressure pipet, and carefully inserted under the renal capsule. This method has been highly successful in experimental islet transplantation, and is also very efficient for analysis of teratoma formation from hES cells. Grafts were removed, fixed and stained with hematoxylin and eosin. Pluripotency was assessed in vitro by assessing gene expression following embryoid body formation as described.

Alternatively, pluripotency was assessed in vivo by examining teratoma formation 8 weeks after transplanting the hES cells under the renal capsule of nude mice as previously described for analysis of endocrine pancreatic progenitor cell differentiation.²⁰ Grafts were removed, fixed, and stained with hematoxylin and eosin. Pluripotency was assessed in vitro by analyzing gene expression in embryoid bodies derived from hES cells and cultured for 17 days.

Proliferation assay to quantify rates of proliferation under different culture conditions: Cells were cultured in 6 well plates on laminin in the presence of either FGF2-supplemented CM from feeder layers or activin A, KGF, NIC, or a combination of these 3 factors. Cultures were pulsed with 1 μCi/ml [methyl ³H] thymidine (specific activity 6.7 Ci/mmol; MP Biomedicals, Irvine Calif.; www.mpbio.com) in newly replenished medium. After 16 hours, cells were harvested and thymidine incorporation into cells was quantified as previously described.²³ Briefly, DNA content was measured fluoremetrically, and incorporation of ³H thymidine was determined by liquid scintillation counting of trichloracetic acid precipitates of the sonicated cells. Statistical significance of observed differences was determined by analysis of variance and Fischer's protected least significance difference test with a 95% level as the limit of significance using Statview IV (Abacus Concepts, Berkeley Calif.).

Flow cytometric analysis: Flow cytometry was used to quantify undifferentiated cells under different culture conditions. Cells were harvested using a 60 minute collagenase treatment followed by shearing into a single cell suspensions and filtering using a 70 micron cell filter. Single cells from each condition were labeled with mouse anti TRA-1-60 or mouse IgM (for controls) and FITC-conjugated donkey anti-mouse IgM (Jackson Immunoresearch). Cells were analyzed using a Becton Dickinson FACScan, and cell-surface antigen expression quantitated using CellQuest software.

Two-dimensional electrophoresis: Unfractionated conditioned media from mEFs grown alone or as a feeder layer with hES cells was assayed by isoelectric focusing and electrophoresis in the second dimension as previously described.²⁴ The sample was transferred to nitrocellulose and blotted with rabbit anti porcine activin antibody, a generous gift from Dr Sunichi Shimisaki, UCSD.

Western blotting: Western blotting was used for phosphorylated Smad2, and was performed on lysates of HSF6 cells as previously described.⁴ Cells cultured in the presence of activin A were lysed in detergent containing buffer supplemented with vanadate (10 μM) and microcystin (1 μM) and first blotted with phospho-Smad2 (ser465/467) antibody (Cell Signaling, cellsignal.com). Blots were then stripped and reprobed with Smad2 antibody (Cell Signaling).

Karyotype: Karyotype analysis was performed in our laboratory or by the UCSD Cytogenetics Laboratory using standard methods (G-banding). At least 15 cells were examined from each sample.

Description of Results Presented in the Figures:

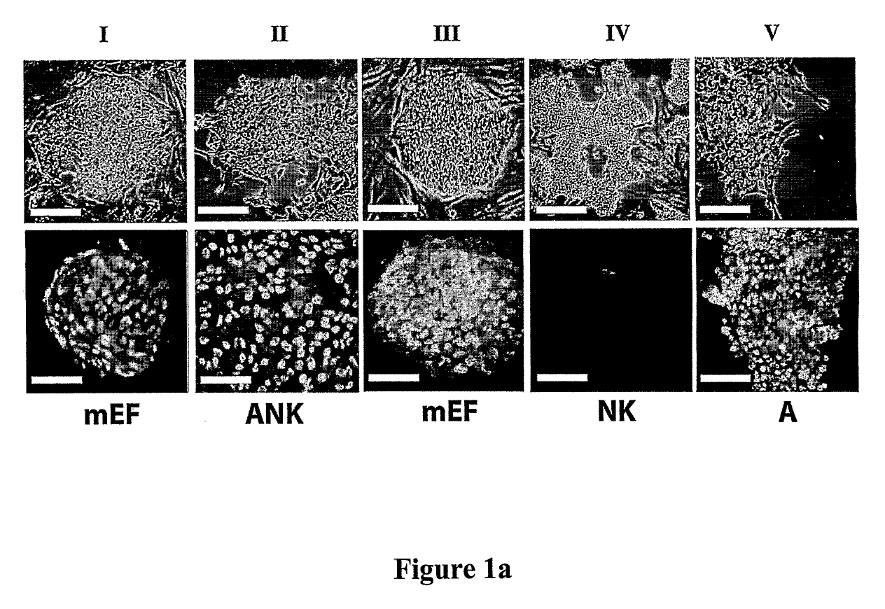

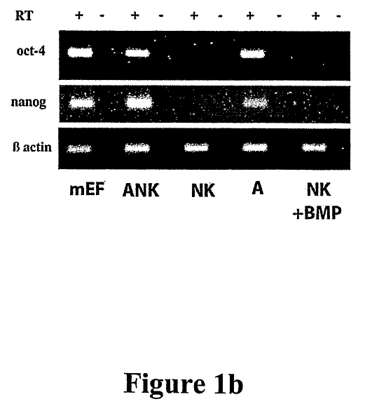

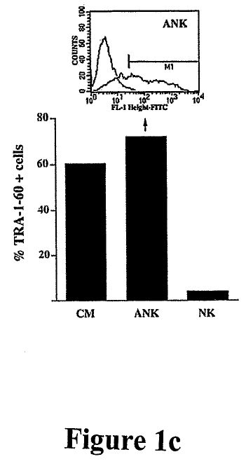

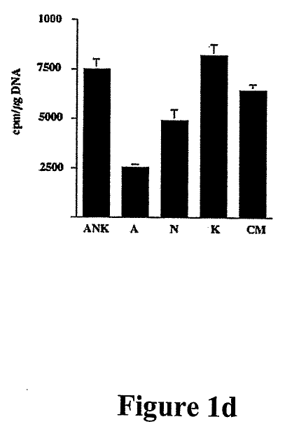

HSF6 cells were used to determine the effect of Activin A on cell growth, morphology, and differentiation status on stem cells. The experiments relating to this Example were performed as described above, as is a discussion and summary of the results obtained from the experiments. The results of the experiments are depicted in the various parts and panels of FIG. 1.

FIG. 1 shows differentiation of hES cells in the absence of activin A. More specifically, FIG. 1a shows the morphology and differentiation state of HSF6 cells observed by phase contrast microscopy (upper layer), and immunohistochemistry (lower layer). For immunohistochemical analysis, antibodies against the human stem cell markers TRA-1-60 (cytoplasmic) and Oct-4 (nuclear) were used. Panel I shows that HSF6 cells cultured on mEFs show typical colony formation, with uniform staining for stem cell markers. Panel II shows that HSF6 cells cultured on laminin in the presence of activin A, NIC, and KGF (ANK), grow as irregular monolayers, with larger cell size than when grown on mEFs, but show robust staining for TRA-1-60 and Oct-4, proof of their undifferentiated state. Panel III shows that cells from Panel II, when put back on mEFs, resume colony morphology after 1 week. Panel IV shows that cells from Panel II, when grown in the absence of activin A (NK) for 1 week, show distinct change in morphology and phenotype, with no staining for TRA-1-60 and very little staining for Oct-4, indicating differentiation. Panel V shows that cells from Panel II grown in the absence of KGF and NIC for 1 week (A) show no change in phenotype. However, proliferation was reduced and they could not be passaged further. Magnification bar=100 μM

FIG. 1b shows semi-quantitative RT-PCR (26 cycles) of hES cells for oct-4 and nanog under a variety of culture conditions on mEFs (lane 1) or on laminin (lane 2-5). Expression of stem cell markers was lost in cells cultured for 1 week on laminin in the absence of activin (lane 3, 5), indicating the need for activin to maintain the undifferentiated phenotype. mEF=mouse feeder layers, ANK=activin A+NIC+KGF, NK═NIC+KGF, BMP=BMP-4, +/−: reverse transcriptase.

FIG. 1c shows a representative experiment of comparison of cell surface-antigen expression using FACS. Single cell suspensions from different culture conditions were immunostained for TRA-1-60 and analyzed using a Becton Dickinson FacScan and CellQuest software. Upper panel: Flow cytometric analysis of cells cultured with ANK stained with mouse anti-TRA-1-60 or mouse IgM (control). Lower panel: Comparison of percentage of cells expressing TRA-1-60 under different conditions. (CM=conditioned medium, ANK=activin A+NIC+KGF, NK=NIC+KGF. Cells were cultured with ANK for 20 passages, with NK for 1 week and in CM for <1 week (to remove contaminating mEFs).

FIG. 1d shows a representative experiment of comparison of proliferation of hES cells in the presence of activin A (A), NIC (N), KGF (K) or a combination of all 3 (ANK), and in FGF2 supplemented CM. Quadruplicate wells for each condition were pulsed for 16 hours with ³H thymidine. Proliferation rate was significantly reduced in activin treated cells compared to all other treatments, and proliferation rate in the presence of KGF was similar to ANK and significantly higher than CM, indicating a role for KGF in replication of hES cells. n=4; p<0.0001 ANK vs. A, K vs. A, CM vs. A

p<0.005 ANK vs. N, N vs. A, K vs. N. p<0.05 K vs. CM. n.s. ANK vs. K, ANK vs. CM, N vs. CM.

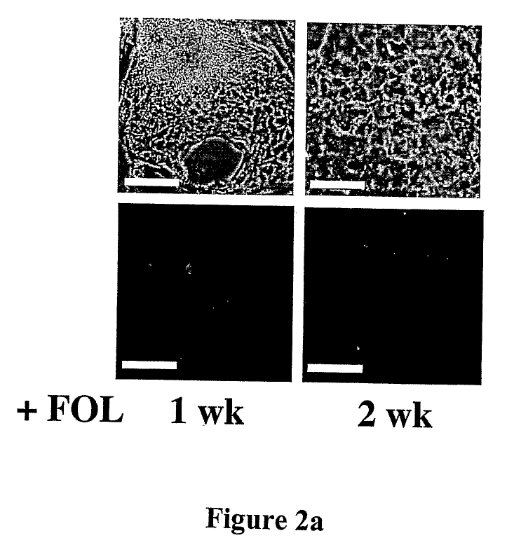

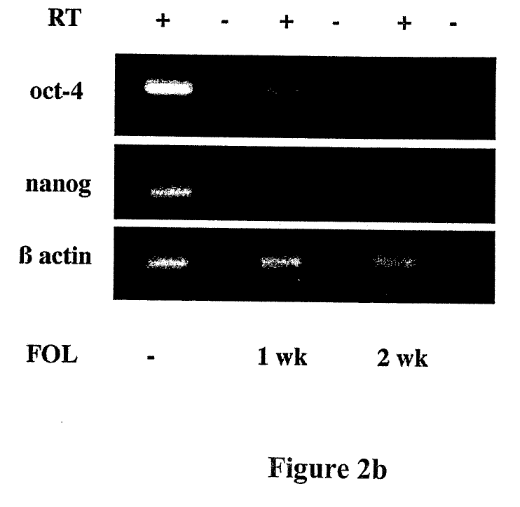

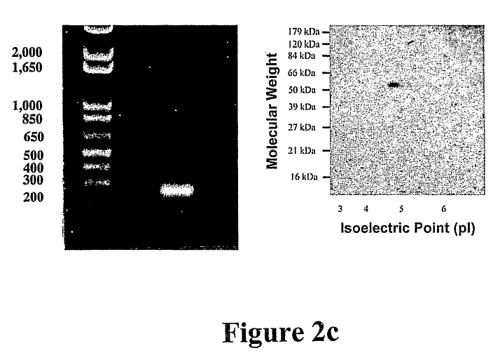

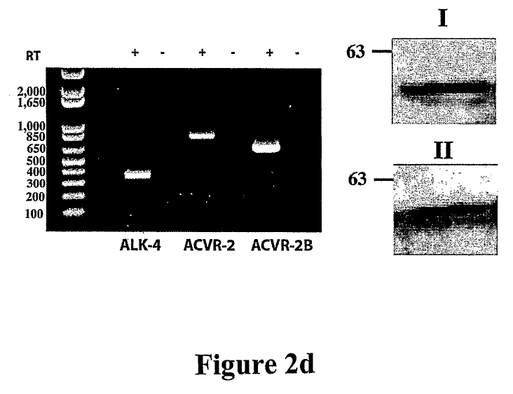

FIG. 2 shows the effect of activin/follistatin on mEF maintenance of pluripotency in HSF6 cells. More specifically, FIG. 2a shows HSF6 cells from FIG. 1 Panel I, cultured on mEFs in the presence of follistatin for 1 week (left panels) and 2 weeks (right panels). After 1 week, colonies showed distinct morphologic changes (upper panel) and lost staining for TRA-1-60 (cytoplasmic), with reduced staining for Oct-4 (nuclear) (lower panel). After 2 weeks in the presence of follistatin, colonies continued to grow but lost their defined shape and Oct-4 immunoreactivity had completely disappeared, indicating differentiation. Magnification bar=100 μM.

FIG. 2b shows semi-quantitative RT-PCR (26 cycles) of HSF6 cells on mEFs for oct-4 and nanog in the presence and absence of follistatin. Expression of stem cell markers was absent (nanog) or markedly diminished (oct-4) in cells cultured for 1 week on mEFs in the presence of follistatin, and completely lost after 2 weeks culture, indicating differentiation. RT=reverse transcriptase.

FIG. 2c shows identification of activin A transcripts in mEFs derived from CF-1 mice and precursor protein in mEF conditioned media using RT-PCR and Western blots. Left panel: RT-PCR showing activin A expression, PCR product size is 262 bp; +/−: reverse transcriptase. Right panel: Western blot showing activin A precursor protein. Samples were analyzed by two-dimensional electrophoresis, Western blotted using anti-activin antibodies.

FIG. 2d shows identification of activin pathway signaling components in HSF6 cells. Left panel: type 1 receptor ALK-4 and type II receptors ACVR-2 and ACVR-2B transcripts in HSF6 cells. PCR product sizes are 346 bp, 783 bp and 611 bp respectively; +/−: reverse transcriptase. Right panel: Western blot using anti Smad2 antibodies showing phospho-Smad 2 in HSF6 cells grown in the presence of Activin A. Smad-2 MW=60 kDa. Cells were lysed in detergent containing buffer supplemented with vanadate (10 μM) and microcystin (1 μM). Blots were probed with anti-phospho-Smad2 (ser/465/467)(panel I), then stripped and reprobed with anti-Smad2 (panel II).

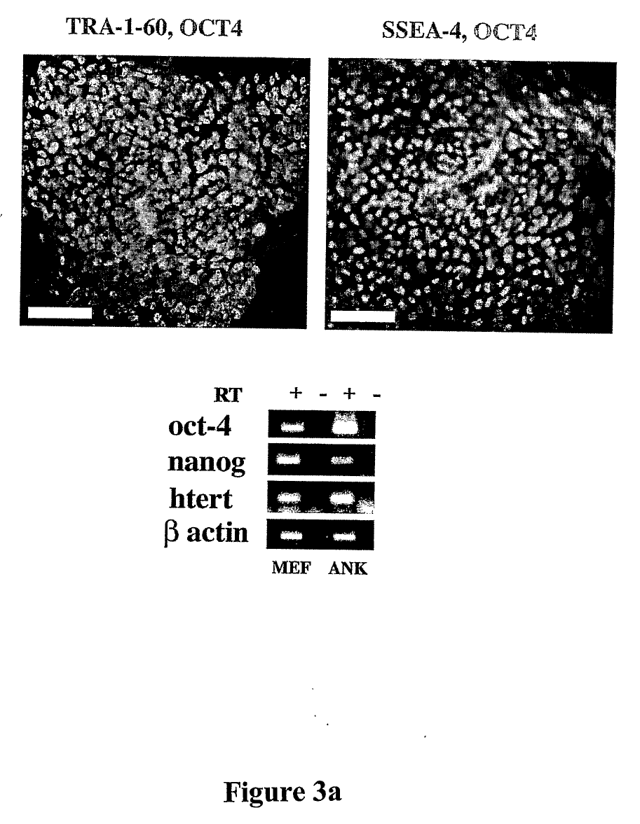

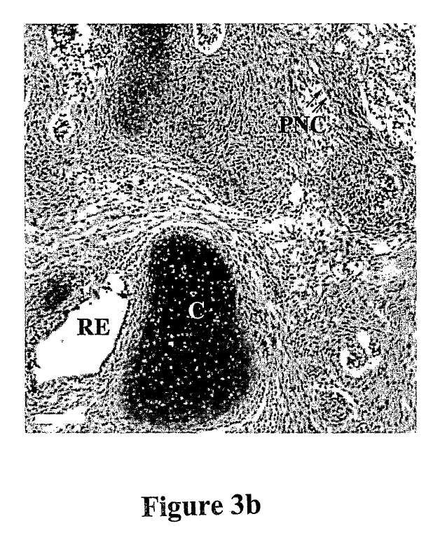

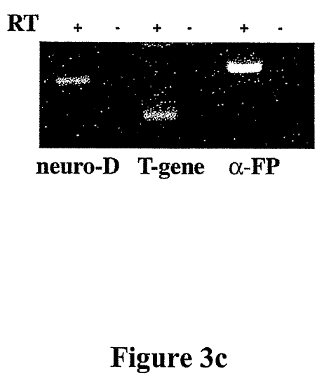

FIG. 3 shows long-term maintenance of pluripotency in hES cells cultured with activin A NIC and KGF. More specifically, FIG. 3a shows analysis of stem cell markers in HSF6 cells cultured in the presence of activin A, KGF and NIC for 20 passages. Upper panel: immunohistochemical analysis shows robust staining for TRA-1-60, SSEA-4 (red) and Oct-4 (green). Magnification bar-200 μM. Lower panel: RTPCR analysis for oct-4, nanog (26 cycles) and hTERT (35 cycles; product=114 bp). For comparison cells cultured on mEFs for a comparable number of passages were analyzed in the same assay, indicating comparable levels of expression of all markers.

FIG. 3b shows teratoma formation in nude mice. Representative histology of HSF6 cells cultured in the presence of activin A, KGF and NIC transplanted under the renal capsule of nude mice. After 8 weeks, kidneys were removed, and teratomas showing evidence of all 3 cell layers were observed. C chondrocytes (mesoderm), PNC=perineural (Schwann) cells (ectoderm), RE=respiratory epithelium (endoderm). Magnification bar=100 μM.

FIG. 3c shows RT-PCR analysis of lineage specific markers in embryoid bodies derived from hES cells cultured in the presence of activin A, KGF and NIC. It shows RNA expression of all cell types. Neuro D=ectoderm, T gene=mesoderm; α-FP=endoderm, +/−: reverse transcriptase.

Summary of the Example

In the present study, we show that hES cells grown on laminin in the presence of activin A, nicotinamide (NIC), and keratinocyte growth factor (KGF) remain undifferentiated during continuous growth over 20 passages

In initial experiments, we sought to develop media to culture hES cells that would direct differentiation into pancreatic endocrine lineage. For cell adhesion we used laminin 1, based on the high levels of α6 β1 expression in hES cells.⁶ A cocktail of various growth factors and chemicals previously shown to modulate cellular growth and differentiation in human fetal pancreatic cells was tested. Surprisingly, hES cells cultured for several weeks under these conditions showed no change in cell morphology. Subsequently, each factor was sequentially eliminated and pluripotency assessed by the expression of known markers for human stem cells: TRA-1-60, nanog, and Oct-4 (data not shown). When the combination of growth factors and chemicals that maintained hES cells replicating in an undifferentiated state was narrowed to Activin A, NIC, and KGF, the experiments were repeated with each of these growth factors alone or in various combinations. Staining of the cultures containing all three factors (A,N,K) was uniform for the stem cell markers TRA-1-60 and Oct-4 (FIG. 3a, Panel II), and comparable to the staining for cells on feeder layers (FIG. 3a, Panel I) or in CM from mEFs (not shown). Robust gene expression of oct-4 and nanog was also observed by RT-PCR in the cell monolayers, with levels comparable to those obtained in colonies growing on feeder layers (FIG. 1b).

A hallmark of stem cells and “stemness” is clustered growth. Interestingly, hES cell appearance gradually changed from the usual tight colony formation to an irregular monolayer of uniformly shaped cells. The cells appeared larger than those observed in the original colonies (FIG. 1a, Panel II). With continuous growth, they eventually formed a continuous monolayer and mounded up in the dish. However these changes were reversible; when cells were placed back on feeder layers they gradually resumed the colony formation similar to that previously observed on feeder layers (FIG. 1a Panel III).

Removal of Activin from the growth medium resulted in the rapid change in cell morphology to a differentiated phenotype (FIG. 1a, Panel IV); after 1 week without Activin A, the cells no longer expressed nanog (FIG. 1b), with concomitant loss of immunoreactive TRA-1-60 (FIG. 1a, Panel IV) and reduced levels of Oct-4 protein (FIG. 1a, Panel IV) and message (FIG. 1b). The immunohistochemical and RT-PCR data were validated by quantitation of cell-surface antigen expression by flow cytometry. Consistent with a previous report of TRA-1-60 expression in the ES cell lines H7 and H14,²⁵ 60.3% of HSF6 grown on laminin in the presence of mEF conditioned medium expressed TRA-1-60. Of cells grown in the defined medium, 45.96% expressed the antigen at passage 2, 60.46% at passage 10, and 71.9% at passage 20, a similar pattern of expression to the parent cells (FIG. 1c). In contrast, when activin was removed from the culture for 1 week the level of expression was reduced to 3.9% (FIG. 1c).

Cells cultured in the defined growth medium were examined for markers of pluripotency up to and including passage 20 and found to express all markers tested: TRA-1-60, SSEA-4, Oct-4 (immunohistochemical analysis), and oct-4 nanog, and telomerase reverse transcriptase (htert) (RT-PCR) (FIG. 3a).

Removal of KGF and NIC from the medium had a different effect; the cells maintained their undifferentiated phenotype (FIG. 1a, Panel V, and FIG. 1b). However there was a significant difference in cell proliferation when cultured with activin, KGF, or NIC alone compared to the combination of the 3 factors (A,N,K). Cells cultured with activin alone did not differentiate (A: FIG. 1a, Panel V; FIG. 1b); however, their proliferation rate was significantly reduced compared to those cultured with the combination (ANK), or with KGF or NIC alone (FIG. 1d, n=4; p<0.0001 vs ANK or KGF, p<0.005 vs NIC). In contrast, there was no statistical difference in the proliferation rate of cells cultured with KGF compared to cells cultured with the combination. Proliferation of cells cultured with NIC alone was intermediate; although significantly less than in the KGF or ANK treated cells (FIG. 1d, n=4; p<0.005), the rate was significantly higher than in activin treated cells (FIG. 1d, n=4; p<0.005). From these data we conclude that activin is important, and perhaps needed, for maintenance of pluripotency, and that KGF, and to a lesser extent NIC help maintain proliferation and continued growth. While cells cultured with activin and KGF (AK) in the absence of NIC remained undifferentiated and grew successfully in the short term, their growth was suboptimal over several passages compared to those cultures which included NIC (data not shown), possibly due to its documented anti-apoptotic effect.²⁶ Therefore, NIC was included in the growth factor combination used during the 20 passages. Importantly, in addition to maintaining markers of undifferentiated cells for 20 passages, these cells also retained a normal karyotype (data not shown).

We next explored whether another member of the transforming growth factor-β (TGFβ) superfamily could maintain pluripotency. Like activin, bone morphogenetic proteins (BMPs) are secreted proteins that regulate numerous cellular responses,²⁷ including differentiation of hES cells into trophoectoderm.²⁸ In addition to its role in differentiation, BMP-4 has also been shown to maintain pluripotency in mES cells.¹² In contrast, when activin A was replaced with BMP-4 in the medium, the hES cells were unable to maintain their undifferentiated phenotype and a complete loss of expression for nanog and oct-4 occurred after 1 week (FIG. 1b). In mES cells BMP-4 plays a paradoxical role in both maintenance of pluripotency and differentiation,^{12,13} most likely due to interactions with other growth factors present at particular stages of development and to different concentrations of the peptide to which the cells are exposed.²⁹ A similar situation may occur with activin A and hES cells, as activin A-induced differentiation of hES cells under certain conditions has already been shown.³⁰

Activin A has been identified in a wide variety of tissues as an autocrine or paracrine regulator of diverse biological functions.^{8,10} Significantly, we found high expression of activin A transcripts in mEFs and secreted activin A precursor protein in the conditioned medium from mEFs (FIG. 2c). In addition HSF6 cells express high levels of both type I and type II activin receptors and robust Smad2 phosphorylation (FIG. 2d). Moreover, after 2 weeks in the presence of the activin inhibitor follistatin, the HSF6 cells grown on mEFs differentiated, completely losing the ES markers TRA-1-60, Oct-4, and nanog (FIG. 2a-b), similar to the effect seen with removal of activin A from the defined medium (FIG. 1a-b). FS-288, the isoform of follistatin used in these experiments, has extremely high affinity for activin A, a lower affinity for members of the BMP family, and does not bind TGFβ.^{10,31} We have already shown that BMP-4 is ineffective in maintaining pluripotency in hES cells; thus it is likely that the differentiation we see results from specific activin/follistatin interactions.

Activin A has been implicated in differentiation of mES into mesoderm¹³ of human pancreatic precursor cells into β cells,¹⁴ inhibition of neural differentiation^{15,16} and, more recently, induction of endoderm in hES cells. ¹⁷ This, however, is the first documentation of the presence of activin in conditioned medium from mEFs, and its subsequent novel role in the maintenance of stem cells in the undifferentiated state. We have detected expression of several Wnts in hES cells (data not shown). Therefore our findings, taken with the recent report of maintenance of hES cell pluripotency through activation of Wnt signaling⁵ may help elucidate a defined molecular pathway for maintenance of pluripotency in hES cells.

Further proof of the pluripotency of the hES cells maintained in activin A enriched medium was provided by teratoma formation in vivo. After transplantation of the hES cells under the kidney capsule in nude mice, the grafts showed evidence of ectodermal, endodermal, and mesodermal structures (FIG. 3b). In addition, lineage specific gene expression profiles obtained by RT-PCR on 17 day old embryoid bodies derived also from cells cultured in the presence of activin A showed a similar pattern of expression for all 3 embryonic cell layers (FIG. 3c). These data show that maintenance of hES in medium containing activin A allows the maintenance of pluripotency without the need for coculture with other foreign or human cells.

The identification of activin A as a key factor in mediating these cellular events will help to unravel the biochemical pathways responsible for “stemness”. An increased efficiency in the generation and culture of human stem cells for potential clinical applications is timely, given the recent report of 17 newly derived stem cell lines available for non-federal supported research.¹⁸ The findings reported here will facilitate the derivation of new human embryonic cell lines without the use of animal or human feeder layers.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

The following references, along with any others cited herein, are incorporated by reference herein in their entireties.
1. Smith, A. G. Embryo-derived stem cells: of mice and men. Annu Rev Cell Dev Biol 17, 435-462 (2001).
2. Thomson, J. A. et al. Embryonic stem cell lines derived from human blastocysts. Science 282, 1145-1147. (1998).
3. Reubinoff, B. E., Pera, M. F., Fong, C. Y., Trounson, A. & Bongso, A. Embryonic stem cell lines from human blastocysts: somatic differentiation in vitro. Nat Biotechnol 18, 399-404. (2000).
4. Humphrey, R., Beattie G M, Lopez, A D, King C C, Bucay N, Hayek A Maintenance of pluripotency in human embryonic stem cells is STAT3 independent. Stem Cells 22, 522-530 (2004).
5. Sato, N., Meijer, L., Skaltsounis, L., Greengard, P. & Brivanlou, A. H. Maintenance of pluripotency in human and mouse embryonic stem cells through activation of Wnt signaling by a pharmacological GSK-3-specific inhibitor. Nat Med 10, 55-63 (2004).
6. Xu, C. et al. Feeder-free growth of undifferentiated human embryonic stem cells. Nat Biotechnol 19, 971-974. (2001).
7. Richards, M., Fong, C. Y., Chan, W. K., Wong, P. C. & Bongso, A. Human feeders support prolonged undifferentiated growth of human inner cell masses and embryonic stem cells. Nat Biotechnol 20, 933-936. (2002).
8. Vale, W. et al. Purification and characterization of an FSH releasing protein from porcine ovarian follicular fluid. Nature 321, 776-779 (1986).
9. Ling, N. et al. Pituitary FSH is released by a heterodimer of the beta-subunits from the two forms of inhibin. Nature 321, 779-782 (1986).
10. Luisi, S., Florio, P., Reis, F. M. & Petraglia, F. Expression and secretion of activin A: possible physiological and clinical implications. Eur J Endocrinol 145, 225-236 (2001).
11. Shav-Tal, Y. & Zipori, D. The Role of Activin A in Regulation of Hemopoiesis. Stem Cells 20, 493-500 (2002).
12. Ying, Q. L., Nichols, J., Chambers, I. & Smith, A. BMP induction of 1d proteins suppresses differentiation and sustains embryonic stem cell self-renewal in collaboration with STAT3. Cell 115, 281-292 (2003).
13. Johansson, B. M. & Wiles, M. V. Evidence for involvement of activin A and bone morphogenetic protein 4 in mammalian mesoderm and hematopoietic development. Mol Cell Biol 15, 141-151 (1995).
14. Demeterco, C., Beattie, G. M., Dib, S. A., Lopez, A. D. & Hayek, A. A role for activin A and betacellulin in human fetal pancreatic cell differentiation and growth. J Clin Endocrinol Metab 85, 3892-3897. (2000).
15. Hashimoto, M. et al. Activin/EDF as an inhibitor of neural differentiation. Biochem Biophys Res Commun 173, 193-200 (1990).
16. Harland, R. Neural induction. Curr Opin Genet Dev 10, 357-362 (2000).
17. Levenberg, S. et al. Differentiation of human embryonic stem cells on three-dimensional polymer scaffolds. Proc. Natl. Acad. Sci. 100, 12741-12746 (2003).
18. Cowan, C. A. et al. Derivation of Embryonic Stem-Cell Lines from Human Blastocysts. N Engl J Med 350:1353-1356 (2004).
19. Abeyta M, C. A., Rodriguez, R., Bodnar, M., Reijo Pera R., Firpo, M. T Unique gene expression signatures of independently derived human embryonic stem cell lines. Human Molecular Genetics 13, in press (2004).
20. Movassat, J., Beattie, G. M., Lopez, A. D., Portha, B. & Hayek, A. Keratinocyte growth factor and beta-cell differentiation in human fetal pancreatic endocrine precursor cells. Diabetologia 46, 822-829 (2003).
21. Hayek, A. & and Beattie, G. M. Experimental Transplantation of Human Fetal and Adult Pancreatic islets. Journal of Clinical Endocrinology and Metabolism 82 No 8, 2471-2475 (1997).
22. Carbonneau, H. and Tonks, Annu. Rev. Cell Biol. 8:463-93, 1992.
23. Hayek A, Beattie G M, Cirulli V, et al. Growth factor/matrix-induced proliferation of human adult beta-cells. Diabetes 44:1458-1460 (1995).
24. King C C, Newton A C. The adaptor protein grb14 regulates the localization of 3-phosphoinositide-dependent kinase-1. J Biol Chem 279:37518-37527 (2004).
25. Henderson J K, Draper J S, Baillie H S, et al. Preimplantation human embryos and embryonic stem cells show comparable expression of stage-specific embryonic antigens. Stem Cells 20:329-337 (2002).
26. Beattie G M, Leibowitz G, Lopez A D, et al. Protection from cell death in cultured human fetal pancreatic cells. Cell Transplant 9:431-438 (2000).
27. Itoh S, Itoh F, Goumans M J, et al. Signaling of transforming growth factor-beta family members through smad proteins. Eur J Biochem 267:6954-6967 (2000).
28. Xu R H, Chen X, Li D S, et al. Bmp4 initiates human embryonic stem cell differentiation to trophoblast. Nat Biotechnol 20:1261-1264 (2002).
29. Gumienny T L, Padgett R W. The other side of tgf-beta superfamily signal regulation: Thinking outside the cell. Trends Endocrinol Metab 13:295-299 (2002).
30. Miyazawa K, Shinozaki M, Hara T, et al. Two major smad pathways in tgf-beta superfamily signalling. Genes Cells 7:1191-1204 (2002).
31. Sidis Y, Tortoriello D V, Holmes W E, et al. Follistatin-related protein and follistatin differentially neutralize endogenous vs. Exogenous activin. Endocrinology 143: 1613-1624 (2002).

1. A method for maintenance of an undifferentiated stem cell, said method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state for a sufficient amount of time to achieve a desired result.

2. The method of claim 1, wherein the method comprises exposing said cell to two or more of a TGFβ family member, an FGF family member, and NIC.

3. The method of claim 1, wherein the method comprises exposing said cell to a TGFβ family member, an FGF family member, and NIC.

4. The method of claim 1, wherein the TGFβ family member is Activin A.

5. The method of claim 1, wherein the FGF family member is keratinocyte growth factor (KGF).

6. The method of claim 1, wherein said exposing results in growth of said cell.

7. The method of claim 1, wherein said exposing is repeated at least one time.

8. The method of claim 1, wherein said stem cell is a mammalian stem cell.

9. The method of claim 1, wherein said stem cell is a human stem cell.

10. The method of claim 1, wherein said stem cell is an embryonic stem cell.

11. The method of claim 1, wherein the desired result comprises culturing said stem cell for ten passages or more.

12. The method of claim 1, wherein the desired result comprises culturing said stem cell for thirty passages or more.

13. The method of claim 1, wherein the TGFβ family member shows 30% or greater sequence identity with SEQ ID NO:1.

14. The method of claim 1, wherein the TGFβ family member shows 80% or greater sequence identity with SEQ ID NO:1.

15. The method of claim 1, wherein the TGFβ family member shows 90% or greater sequence identity with SEQ ID NO:1.

16. The method of claim 1, wherein the TGFβ family member shows 95% or greater sequence identity with SEQ ID NO:1.

17. The method of claim 1, wherein the TGFβ family member shows 99% or greater sequence identity with SEQ ID NO:1.

18. The method of claim 1, wherein the FGF family member shows 30% or greater sequence identity with SEQ ID NO:17.

19. The method of claim 1, wherein the FGF family member shows 80% or greater sequence identity with SEQ ID NO:17.

20. The method of claim 1, wherein the FGF family member shows 90% or greater sequence identity with SEQ ID NO:17.

21. The method of claim 1, wherein the FGF family member shows 95% or greater sequence identity with SEQ ID NO:17.

22. The method of claim 1, wherein the FGF family member shows 99% or greater sequence identity with SEQ ID NO:17.

23. A composition comprising a) a culture medium and b) a TGFβ family member, an FGF family member, NIC, or a combination of two or more of these.

24. The composition of claim 23, wherein the TGFβ family member is Activin A.

25. The composition of claim 23, wherein the FGF family member is KGF.

26. The composition of claim 23, further comprising a stem cell.

27. The composition of claim 26, wherein said stem cell is a mammalian stem cell.

28. The composition of claim 26, wherein said stem cell is a human stem cell.

29. The composition of claim 26, wherein said stem cell is an embryonic stem cell.

30. The composition of claim 23, wherein the TGFβ family member shows 30% or greater sequence identity with SEQ ID NO:1.

31. The composition of claim 23, wherein the TGFβ family member shows 80% or greater sequence identity with SEQ ID NO:1.

32. The composition of claim 23, wherein the TGFβ family member shows 90% or greater sequence identity with SEQ ID NO:1.

33. The composition of claim 23, wherein the TGFβ family member shows 95% or greater sequence identity with SEQ ID NO:1.

34. The composition of claim 23, wherein the TGFβ family member shows 99% or greater sequence identity with SEQ ID NO:1.

35. The composition of claim 23, wherein the FGF family member shows 30% or greater sequence identity with SEQ ID NO:17.

36. The composition of claim 23, wherein the FGF family member shows 80% or greater sequence identity with SEQ ID NO:17.

37. The composition of claim 23, wherein the FGF family member shows 90% or greater sequence identity with SEQ ID NO:17.

38. The composition of claim 23, wherein the FGF family member shows 95% or greater sequence identity with SEQ ID NO:17.

39. The composition of claim 23, wherein the FGF family member shows 99% or greater sequence identity with SEQ ID NO:17.

40. A composition comprising a combination of two or more of a) at least one purified TGFβ family member protein, b) at least one purified FGF family member protein, and 3) purified NIC.

41. The composition of claim 40, which is a culture medium for stem cells.

42. The composition of claim 41, wherein the stem cells are mammalian stem cells.

43. The composition of claim 41, wherein the stem cells are human stem cells.

44. The composition of claim 41, wherein the stem cells are embryonic stem cells.

45. The composition of claim 41, wherein the TGFβ family member is Activin A.

46. The composition of claim 41, wherein the FGF family member is KGF.

47. An undifferentiated, pluripotent stem cell derived from a culture that has been passaged at least 10 times.

48. The stem cell of claim 47, wherein the culture has been passaged at least 20 times.

49. The stem cell of claim 47, wherein the culture has been passaged at least 30 times.

50. The stem cell of claim 47, wherein the stem cell has been passaged in the presence of media comprising a TGFβ family member, an FGF family member, or NIC, but not in the presence of conditioned media, feeder cells, or LIF.

51. The stem cell of claim 47, wherein the stem cell is a mammalian stem cell.

52. The stem cell of claim 47, wherein the stem cell is a human stem cell.

53. The stem cell of claim 47, wherein the stem cell is an embryonic stem cell.

54. A stem cell produced by a method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state.

55. A kit comprising two or more of the following:
a) a TGFβ family member,
b) an FGF family member,
c) NIC,
d) a stem cell, and
e) stem cell culture media.

56. The kit of claim 55, wherein the TGFβ family member is Activin A.

57. The kit of claim 55, wherein the FGF family member is KGF.

58. The kit of claim 55, wherein the stem cell is a mammalian stem cell.

59. The kit of claim 55, wherein the stem cell is a human stem cell.

60. The kit of claim 55, wherein the stem cell is an embryonic stem cell.

61. A method for maintenance of an undifferentiated stem cell, said method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state for a sufficient amount of time to achieve a desired result, wherein the stem cell is not also exposed to a feeder cell, conditioned media, or leukemia inhibitory factor.

62. A composition comprising a) a culture medium and b) a TGFβ family member, an FGF family member, NIC, or a combination of two or more of these, wherein said composition does not comprise feeder cells, conditioned media, or LIF.

63. A differentiated cell derived from a stem cell grown or maintained by a method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state.

64. An embryonic stem cell grown or maintained by a method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state.

65. A pharmaceutical composition comprising a stem cell grown or maintained by a method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state.

66. A cell derived from a stem cell grown or maintained by a method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state


SEQUENCE LISTING (57 sequences)

SEQ ID NO: 1 - protein, 116 aa, Homo sapiens [sequence omitted]

SEQ ID NO: 2 - DNA, 351 bp, Homo sapiens
ggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatag

SEQ ID NO: 3 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 4 - protein, 130 aa, Homo sapiens
ARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 5 - protein, 115 aa, Homo sapiens
GLECDGKVNICCKKQFFVSFKDIGWNDWIIAPSGYHANYCEGECPSHIAGTSGSSLSFHSTVINHYACGHSPFANLKSCCVPTKLRPMSMLYYDDGQNIIKKDIQNMIVEECGCS

SEQ ID NO: 6 - protein, 426 aa, Homo sapiens; identical to SEQ ID NO: 3

SEQ ID NO: 7 - protein, 424 aa, Mus musculus
MPLLWLRGFLLASCWIIVRSSPTPGSEGHGSAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 8 - protein, 424 aa, Rattus norvegicus
MPLLWLRGFLLASCWIIVRSSPTPGSEGHGAAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDMGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 9 - protein, 424 aa, Gallus gallus
MPLLWKRGFLLVICWIIVRSSPTPGSEGHSSVADCPSCALTTLSKDVPSSQPEMVEAVKKHILNMLHLRDRPNITQPVPKAALLNATKKLHVGKVGDDGYVEIEDDVGRRAEMNEVVEQTSEIITFAESGTPKKTLHFEISKEGSELSVVEHAEVWLFLKVSKANRSRTKVTIRLFQQQRQPKGNSEAAEDMEDMGLKGERSETLISEKAVDARKSTWHIFPISSSVQRLLDQGQSSLDVRIACDLCQETGASLVLLGKKKKKEDDGEGKEKDGGELTGEEEKEQSHRPFLMMLARHSEDRQHRRRERGLECDGKVNICCKKQFFVSFKDIGWSDWIIAPTGYHANYCEEECPSHIAGTSGSSLSFHSTVINHYRMRGHSPFANLKSCCVPTKLRPMSMLYYDDGQNIIKKDIQNMIVEECGCS

SEQ ID NO: 10 - protein, 425 aa, Bos taurus
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKKKEEEGEGKKRDGEGGAGGDEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 11 - protein, 426 aa, Equus caballus
MPLLWLRGFLLASCWIIVKSSPTPGSEGHSAAPNCPSCALATLPKDVPNAQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRSKVTIRLLQQQKHPQGSSDTREEAEEADLMEERSEQLISEKVVDARKSTWHIFPVSSSIQRLLDQGKSSLDIRIACDQCHETGASLVLLGKKKKKEEEGEGKKKDGGEAGAGVDEEKEQSHRPFLMLQARQSEDHPHRRRRRGLECDGKVNICCKKQFFVSFKDIGWNDWIIAPSGYHANYCEGECPSHIAGTSGSSLSFHSTVINQYRLRGHNPFANLKSCCVPTKLRPMSMLYYDDGQNIIKKDIQNMIVEECGCS

SEQ ID NO: 12 - protein, 424 aa, Sus scrofa
MPLLWLRGFLLASCWIIVRSSPTPGSGGHSAAPDCPSCALATLPKDVPNSQPEMVEAVKKHILNMLHLKKRPDVTQPVPKAALLNAIRKLHVGKVGENGYVELEDDIGRRAEMNELMEQTSEIITFAEAGTARKTLRFEISKEGSDLSVVERAEIWLFLKVPKANRTRTKVSIRLFQQQRRPQGSADAGEEAEDVGFPEEKSEVLISEKVVDARKSTWHIFPVSSSIQRLLDQGKSALDIRTACEQCHETGASLVLLGKKKKKEEEAEGRKRDGEGAGVDEEKEQSHRPFLMLQARQSEEHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 13 - protein, 425 aa, Ovis aries
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISQEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKRKEEEGEGKKRDGEGGAGGDEEKEQSHRPFLMLQARQSEDHPHRRRRRGLECDGKVNICCKKQFYVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 14 - protein, 424 aa, Felis catus
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 15 - protein, 395 aa, Danio rerio
MSPLPLLSGILLLLIRSCSLSAMVTKGSLPMSEQQAGATVCPSCALARFRKGVSESEDEGAQQDVVEAVKRHILNMLHLQERPNITHPVPRAALLNAIRKVHVGRVAKDGSVLIEDEASNRAETEQAEQTEIITFAETGEAPGIVNFLISKEGGEMSVVDQANVWIFLRLPKGNRTRANVNIRLLLQQGAGEKILAEKSVDTRRSGWHTFPASESVQSLLQRGGSTLSLRVSCPLCADARATPVLVSPGGSEREQSHRPFLMAVVRQMDELSLRRRRKRGLECDGKARVCCKRQFYVNFKDIGWNDWIIAPSGYHANYCEGDCASNVASITGNSLSFHSTVISHYRIRGYSPFTNIKSCCVPTRLRAMSMLYYNEEQKIVKKDIQNMIVEECGCS

SEQ ID NO: 16 - protein, 404 aa, Carassius auratus
MSSLTLVNRGTAALRLFVRGLLTHSSREWLSGDGEPDDPVTPCPSCALAQRQKDSEEQTDMVEAVKRHILNMLHLNTRPNVTHPVPRAALLNAIRRLHVGRVGEDGTVEMEEDGGGLGEHREQSEEQPFEIITFAEPGDAPDIMKFDISMEGNTLSVVEQANVWLLLKVAKGSRGKGKVSVQLLQHGKADPGSADGPQEAVVSEKTVDTRRSGWHTLPVSRTVQTLLDGDSSMLSLRVSCPMCAEAGAVPILVPTESNKGKEREQSHRPFLMVVLKPAEEHPHRRSKRGLECDGKIRVCCKRQFYVNFKDIGWSDWIIAPSGYHANYCEGDCPSHVASITGSALSFHSTVINHYRMRGYSPFNNIKSCCVPTRLRAMSMLYYNEEQKIIKKDIQNMIVEECGCS

SEQ ID NO: 17 - protein, 164 aa, Homo sapiens
MCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 18 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 19 - protein, 194 aa, Mus musculus
MRKWILTRILPTLLYRSCFHLVCLVGTISLACNDMSPEQTATSVNCSSPERHTRSYDYMEGGDIRVRRLFCRTQWYLRIDKRGKVKGTQEMKNSYNIMEIRTVAVGIVAIKGVESEYYLAMNKEGKLYAKKECNEDCNFKELILENHYNTYASAKWTHSGGEMFVALNQKGIPVKGKKTKKEQKTAHFLPMAIT

SEQ ID NO: 20 - protein, 194 aa, Canis familiaris
MRKWILTWILPTLLYRSCFHIICLVGTISLACNDMTPEQMATNVNCSSPERHTRSYDYMEGGDIRVRRLFCRTQWYLRIDKRGKVKGTQEMKNSYNIMEIRTVAVGIVAIKGVESEYYLAMNKEGKLYAKKECNEDCNFKELILENHYNTYASAKWTHSGGEMFVALNQKGVPVRGKKTKKEQKTAHFLPMAIT

SEQ ID NO: 21 - protein, 194 aa, Sus scrofa
MRKWILTWILPSLLHRSCFHIICLVGTLSLDCNDMTPEQMATNVNCSSPERHTRSYDYMEGGDIRVRRLFCRTQWYPRIGKRGKVKGTQEMKNNYNIMEIRTVAVGIVAIKGVVSEYYLAMNKEGKLYAKKEYNEDCNFKELILENHYNTYASAKWTHSGGEMFVALNQKGVPVRGKKTKKEQKTAHFLPMAIT

SEQ ID NO: 22 - protein, 194 aa, Rattus norvegicus
MRKWILTRILPTPLYRPCFHLVCLVGTISLACNDMSPEQTATSVNCSSPERHTRSYDYMEGGDIRVRRLFCRTQWYLRIDKRGKVKGTQEMRNSYNIMEIMTVAVGIVAIKGVESEYYLAMNKQGELYAKKECNEDCNFKELILENHYNTSASAKWTHSGGEMFVALNQKGLPVKGKKTKKEQKTAHFLPMAIT

SEQ ID NO: 23 - protein, 194 aa, Ovis aries; identical to SEQ ID NO: 20

SEQ ID NO: 24 - protein, 186 aa, Mustela vison
MRKWILTWILPTLLYRSCFHIICLVGTISLACNDMTPEQMATNVNCSSPERHTRSYDYMEGGDIRVRRLFCRTQWYLRIDKRGKVKGTQEMKNSYNIMEIRTVAVGIVAIKGVESEYYLAMNKEGKLYAKKECNEDCNFKELILENHYNTYASAKWTHSGGEMFVALNQKGVPVRGKKTKKEQKQP

SEQ ID NO: 25 - DNA, 1200 bp, Homo sapiens
acgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaaggaggaatcctgtgttgttatcaggaactaaaaggataaggctaacaatttggaaagagcaagtactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaacccagttccagcagggagatttctttaagtggactgttttctttcttctcaaaattttctttccttttattttttagtaatcaagaaaggctggaaaaactactgaaaaactgatcaagctggacttgtgcatttatgtttgttttaag

SEQ ID NO: 26 - DNA, 585 bp, Mus musculus
atgcgcaaatggatactgacacggatcctgccaactctgctctacagatcatgcttccacctcgtctgtctagtgggcactatatctctagcttgcaatgacatgagtccggagcaaacggctacgagtgtgaactgttccagccccgagcgacacaccagaagttatgactacatggaaggaggggatataagggtgagaagactgttctgtcgcacccagtggtacctgaggattgacaaacgaggcaaagtgaaagggacccaggagatgaagaacagctacaacatcatggaaatcaggaccgtggcagttggaattgtggcaatcaaaggggtggaaagtgaatactatcttgccatgaacaaggaagggaaactctatgcaaagaaagaatgcaatgaggattgcaacttcaaagaactgattctggaaaaccattataacacctatgcatcagctaaatggacacacagcggaggggaaatgttcgttgccttaaatcaaaaggggattcctgtcaaagggaagaaaacgaagaaagaacaaaaaacagcccattttcttcctatggcaataacctaa

SEQ ID NO: 27 - DNA, 686 bp, Canis familiaris
agaggtcaatgacccaggagcaacaatcaactcaagatttaattttcattatgttattcatgaacacccggagcactacactataatgcgcaaatggatactgacatggatcctgccaactttgctctacagatcatgctttcacattatctgtctagtgggcactatatctttagcttgcaatgacatgactccagagcaaatggctacaaatgtgaactgttccagccctgagcgacatacaagaagttatgattacatggaaggaggggatataagagtgagaagactcttctgtcgaacacagtggtatctgaggattgataaacgaggcaaagtcaaagggacccaagagatgaagaacagttacaatatcatggaaatcaggacagtggcagttggaatagtggcaatcaaaggggtggaaagtgaatattatcttgcaatgaataaggaaggaaagctctatgcaaagaaagaatgcaatgaagattgcaacttcaaagaattaattctggaaaaccattacaacacatatgcatcagctaaatggacacacagcggaggagaaatgtttgttgctttaaatcaaaagggggttcctgtaagggggaaaaaaacgaagaaagaacaaaaaacagcccactttcttcctatggcaataacataatcatatatggtatata

SEQ ID NO: 28 - DNA, 690 bp, Sus scrofa
aatctacaattcacagataggaagaggtcagtgacctaggagcaacgatcaactcaagatttattttcattatgttattcatgaacacccggagcactatactataatgcgcaaatggatactgacatggatcctgccaagtttgctccacagatcatgcttccacattatctgtctggtgggcactttatctttggattgcaatgacatgactccagagcaaatggctacaaatgtgaactgttccagccctgagcgacatacaagaagttatgattacatggaaggaggggatataagagtgagaagactcttctgtcgaacacagtggtatccgaggattggcaaacgaggcaaagtcaaagggactcaagagatgaagaacaattacaacatcatggaaatcaggacagtggctgttggaattgtagcaatcaaaggagtggtaagtgaatattatcttgcaatgaacaaggaaggaaaactctatgcaaagaaagaatacaatgaagattgtaacttcaaagaattaattctggaaaaccattacaacacgtatgcatcagctaaatggacacacagtggaggagaaatgtttgttgccttaaatcaaaagggggttcctgtaagagggaaaaaaaccaagaaagaacaaaaaacagcccactttcttcctatggcaataactaa

SEQ ID NO: 29 - DNA, 693 bp, Rattus norvegicus
caatctacaattcacagataggaggaggcccatgacctaggagtagcgatcaactcaaggtccagttctcattatgttattcatggacacccggggcactgctctataatgcgcaaatggatactgacacggatcctgccgactccgctctacagaccgtgcttccacctcgtctgtcttgtgggcaccatatctttagcttgcaatgacatgagtccagagcagacggccacgagcgtgaactgttctagccccgagcgacacacgagaagttatgactacatggaaggaggggatataagggtgaggagactgttctgtcgcacccagtggtacctgaggattgacaaacgaggcaaagtgaaagggacccaggagatgaggaacagctacaacatcatggaaatcatgactgtggcagttggaattgtggcaatcaaaggggtggaaagtgaatactatcttgccatgaacaaacaaggggaactctatgcaaagaaagaatgcaatgaggattgcaacttcaaagaactgattctggaaaaccattacaacacctctgcatcagctaaatggacacacagcggaggggaaatgttcgtggccttaaatcaaaaggggcttcctgtcaaagggaagaaaacgaagaaagaacaaaaaacggcccactttcttcctatggcaataacttaa

SEQ ID NO: 30 - DNA, 622 bp, Ovis aries
ttatgttattcatgaacacccggagcactatactataatgcgcaaatggatactgacatggatcctgccaagtttgctctacagatcatgcttccacattatctgtctagtgggcactatatctttagcttgcaatgacatgactccagagcaaatggctacaaatgtgaactgttccagccccgagcgacatacaagaagttatgattacatggaaggaggagatataagagtgagaagactcttctgtcgaacacagtggtatctgaggattgataaacgaggcaaagtcaaagggactcaagagatgaagaataattacaacatcatggaaatcaggacagtggctgttggaattgtagcaatcaaaggagtggaaagtgaatattaccttgcaatgaacaaggaaggaaaactctatgcaaagaaagaatgtaacgaagactgcaacttcaaagaattaattctggaaaatcattacaacacatatgcatcagctaaatggacacacagtggaggagaaatgtttgttgccttaaattcaaaaggggttccagtaagagggaagaaaacgaagaaagaacaaaaaacagcccactttcttcctatggcaataacttaa

SEQ ID NO: 31 - DNA, 558 bp, Mustela vison
atgcgcaaatggatactgacatggatcctgccaactttgctctacagatcatgctttcacattatctgtctagtgggcactatatctttagcttgcaatgacatgactccagagcaaatggctacaaatgtgaactgttccagccctgagcgacatacaagaagttatgattacatggaaggaggggatataagagtgagaagactcttctgtcgaacacagtggtatctgaggattgataaacgaggcaaggtcaaaggaacccaagagatgaagaacagttacaatatcatggaaatcaggacagtggcagttggaattgtggcaatcaaaggggtggaaagtgaatattatcttgcaatgaataaggaaggaaaactctatgcaaagaaagaatgcaatgaagattgcaacttcaaagaattaattctggaaaaccattacaacacatatgcatcagctaaatggacacacagcggaggagaaatgtttgttgctttaaatcaaaagggggttcctgtaagggggaaaaaaacgaagaaagaacaaaaacagccc

SEQ ID NO: 32 - DNA, 1633 bp, Homo sapiens
tgctccctgacagccacaaacctacagcactgactgcattcagagaggaacctgcaaacaaaacttcacagaaaactttttgttcttgttccagagaatttgctgaagaggagaaggaaaaaaaaaacaccaaaaaaaaaaataaaaaaatccacacacacaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaaca

SEQ ID NO: 33 - DNA, 425 bp, Homo sapiens
gcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaaga

SEQ ID NO: 34 - DNA, 348 bp, Homo sapiens
ggcctggagtgcgacggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactacgcatgcggccatagcccctttgccaacctcaaatcgtgctgtgtgcccaccaagctgagacccatgtccatgttgtactatgatgatggtcaaaacatcatcaaaaaggacattcagaacatgatcgtggaggagtgcgggtgctcctaa

SEQ ID NO: 35 - DNA, 1840 bp, Homo sapiens
tccacacacacaaaaaacctgcgcgtgaggggggaggaaaagcagggcctttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaacaaaaaaaaaaacaaaaaaaaacaaaagtaaattaaaaacaaacctgatgaaacagatgaaacagatgaaggaagatgtggaaatcttagcctgccttagccagggctcagagatgaagcagtgaagagacagattgggagggaaagggagaatggtgtaccctttatttcttctgaaatcacactgatgacatcagttgtttaaacggggtattgtcctttccccccttgaggttcccttgtgagcttgaatcaaccaatctgatctgcagtagtgtggactagaacaacccaaatagcatctagaaagccatgagtttgaaagggcccatcacaggcactttcctagcctaat

SEQ ID NO: 36 - DNA, 20 bp, Artificial Sequence
  Description of Artificial Sequence Synthetic primer
cgcaccactggcattgtcat

SEQ ID NO: 37 - DNA, 20 bp, Artificial Sequence
  Description of Artificial Sequence Synthetic primer
ttctccttgatgtcacgcac

SEQ ID NO: 38 - DNA, 19 bp, Artificial Sequence
  Description of Artificial Sequence Synthetic primer
gagcaaaacccggaggagt

SEQ ID NO: 39 - DNA, 19 bp, Artificial Sequence
  Description of Artificial Sequence Synthetic primer
ttctctttcgggcctgcac

SEQ ID NO: 40 - DNA, 20 bp, Artificial Sequence
  Description of Artificial Sequence Synthetic primer
gcttgccttgctttgaagca

SEQ ID NO: 41 - DNA, 20 bp, Artificial Sequence
  Description of Artificial Sequence Synthetic primer
ttcttgactgggaccttgtc

SEQ ID NO: 42 - DNA, 19 bp, Artificial Sequence
  Description of Artificial Sequence Synthetic primer
cttgaagaagagacccgat

SEQ ID NO: 43 - DNA, 19 bp, Artificial Sequence
  Description of Artificial Sequence Synthetic primer
cttctgcacgctccactac

SEQ ID NO: 44 - DNA, 20 bp, Artificial Sequence
  Description of Artificial Sequence Synthetic primer
gagactatcactgctcagga

SEQ ID NO: 45 - DNA, 20 bp, Artificial Sequence
  Description of Artificial Sequence Synthetic primer
gataagcccttgcaaagcgt

SEQ ID NO: 46 - DNA, 19 bp, Artificial Sequence
  Description of Artificial Sequence Synthetic primer
caaccaccgctggaagtac

SEQ ID NO: 47 - DNA, 19 bp, Artificial Sequence
  Description of Artificial Sequence Synthetic primer
ccgctatgaactgggtctc

SEQ ID NO: 48 - DNA, 20 bp, Artificial Sequence
  Description of Artificial Sequence Synthetic primer
agaacctgtcacaagctgtg

SEQ ID NO: 49 - DNA, 20 bp, Artificial Sequence
  Description of Artificial Sequence Synthetic primer
gacagcaagctgaggatgtc

SEQ ID NO: 50 - DNA, 19 bp, Artificial Sequence
  Description of Artificial Sequence Synthetic primer
cacgtgtgagacagatggg

SEQ ID NO: 51 - DNA, 19 bp, Artificial Sequence
  Description of Artificial Sequence Synthetic primer
ggcggttgtgatagacacg

SEQ ID NO: 52 - DNA, 19 bp, Artificial Sequence
  Description of Artificial Sequence Synthetic primer
gggagctgctgcaaagttg

SEQ ID NO: 53 - DNA, 19 bp, Artificial Sequence
  Description of Artificial Sequence Synthetic primer
ccacatcaacactggtgcc

SEQ ID NO: 54 - DNA, 19 bp, Artificial Sequence
  Description of Artificial Sequence Synthetic primer
caccatcgagctcgtgaag

SEQ ID NO: 55 - DNA, 19 bp, Artificial Sequence
  Description of Artificial Sequence Synthetic primer
gagcccttgtcatggaagg

SEQ ID NO: 56 - DNA, 20 bp, Artificial Sequence
  Description of Artificial Sequence Synthetic primer
cagctcccatttcatcagca

SEQ ID NO: 57 - DNA, 19 bp, Artificial Sequence
  Description of Artificial Sequence Synthetic primer
cgacatccctgcgttcttg